(19) United States
(12) Patent Application Publication
(10) Pub. No.: US 2023/0128187 A1
(43) Pub. Date: Apr. 27, 2023

(54) BACTERIAL CONSORTIUM COMPRISING AT LEAST ONE BACILLUS AND LACTOBACILLUS STRAIN FOR GLUTEN DEGRADATION

(71) Applicant: Evonik Operations GmbH, Essen (DE)

(72) Inventors: Bodo SPECKMANN, Kahl (DE); Michael SCHWARM, Alzenau (DE); Stefan PELZER, Guetersloh (DE); Thomas BERNGRUBER, Hamm (DE); Marco GOBBETTI, Bozen-Bolzano (IT); Raffaella DI CAGNO, Bozen-Bolzano (IT)

(73) Assignee: Evonik Operations GmbH, Essen (DE)

(21) Appl. No.: 17/788,239

(22) PCT Filed: Nov. 27, 2020
(86) PCT No.: PCT/EP2020/083770

§ 371 Date: Jun. 22, 2022

(30) Foreign Application Priority Data
Dec. 23, 2019 (EP) 19219287.0

(51) Int. Cl. A61K 35/747; C12N 1/20; A61P 1/14; A61K 35/742; A61K 35/744; A23L 33/135; A23K 10/18; A23K 50/40

(52) U.S. Cl. CPC A61K 35/747; C12N 1/20; A61P 1/14; A61K 35/742; A61K 35/744; A23L 33/135; A23K 10/18; A23K 50/40; A61K 2035/115

(57) ABSTRACT

A preparation comprising probiotic strains belonging to the genera Bacillus sp., Lactobacillus sp., optionally Pediococcus sp. as viable cells or cytoplasmic extract thereof, and proteases, wherein the strains can degrade peptide sequences of gliadins. The preparation can be used to produce gluten-free foods from gluten-containing cereals or to treat gluten-related disorders.

### CROSS-REFERENCE TO RELATED APPLICATIONS

The present application is a 35 U.S.C. § 371 national stage application of International patent application PCT/EP2020/083770, filed Nov. 27, 2020, which is based on and claims the benefit of priority to European Application 19219287.0, filed Dec. 23, 2019.The entire contents of these applications are incorporated herein by reference.

### REFERENCE TO A SEQUENCE LISTING

In accordance with 37 CFR § 1.52(e)(5), the present specification makes reference to a Sequence Listing submitted electronically as a .txt file named “542420US_ST25”. The .xml file was generated on Dec. 23, 2022 and is 4,096 bytes in size. The entire contents of the Sequence Listing are hereby incorporated by reference.

The current invention concerns preparations comprising probiotic strains belonging to the genera Bacillus sp., Lactobacillus sp., and Pediococcus sp. as viable cells or cytoplasmic extract thereof, and proteases and their use for safe gluten degradation in humans and during the production of foods for humans and animals.

Gluten is the main protein network of cereals such as wheat, rye, and barley. Gluten includes monomeric α-gliadins, γ-gliadins, Ω-gliadins, which carry peptide sequences with immunogenic and/or toxic potential (the most prominent examples are listed in table 1).

TABLE 1
Immunogenic gliadin peptides

| Gliadin | Position | Sequence |
|---|---|---|
| α9-gliadin | 57-68 | QLQPFPQPQLPY (SEQ ID NO: 1) |
| A-gliadin | 62-75 | PQPQLPYPQPQSFP (SEQ ID NO: 2) |
| Y-gliadin | 134-153 | QQLPQPQQPQQSFPQQQRPF (SEQ ID |
| | | NO: 3) |
| α2-gliadin | 57-89 | LQLQPFPQPQLPYPQPQLPYPQPQLPYPQ |
| | | PQPF (SEQ ID NO: 4) |

Incomplete digestion of gliadins can release these peptides, leading to adverse reactions in susceptible individuals. The literature also shows the release of immunogenic peptides from glutenins, the other protein fraction constituting gluten. The spectrum of gluten-related disorders includes celiac disease (CD), wheat allergy (WA), non-celiac gluten sensitivity (NCGS), and gluten-sensitive irritable bowel syndrome [1]. Though these disorders have pathogenic differences, they show related symptoms and, as they are not curable, are treated by avoidance of gluten/gluten-containing foods. Moreover, various other health conditions (e.g. schizophrenia, atopy, fibromyalgia, endometriosis, obesity, non-specific gastrointestinal symptoms) have been suggested to benefit from gluten avoidance [2]. These facts explain the rise of gluten-free diets (GFD); and such practice also extends to a large and increasing number of healthy, symptom-free people. For example, reportedly 33% of the US population wants to avoid gluten, and 41% of an athlete population reported being on a GFD for more than 50% of the time [3].

Practicing a GFD is however associated with challenges and adverse effects, which need to be considered in a risk and benefit evaluation, especially when there is no clear indication to maintain a GFD, i.e. where gluten avoidance is rather a lifestyle choice than a medical necessity. A GFD is often imbalanced, e.g. due to the avoidance of cereal products, with micronutrient and fiber deficiencies, alongside an excess of calories and an increased content of sugar and saturated fats found in many gluten-replacement foods [4-6]. Potential harms of a GFD therefore include growth/development retardation for children and adolescents, various malnutrition-associated disorders, hyperlipidemia, hyperglycemia, and coronary artery disease [6]. Moreover, long-term adherence to a GFD can cause intestinal microbiome dysbiosis with subsequent adverse health effects [7].

A GFD is at present the only effective therapy for CD, WA, and NCGS patients. Particularly for CD, ingestion of gluten or similar proteins are the trigger for the development and exacerbation of the disease, whereby strict avoidance of gluten ingestion is of critical importance. However, even food products considered or claimed as being gluten-free often contain (trace) amounts of gluten that are above a safe limit of gluten intake (typically <20 ppm for CD patients). To ensure food safety for CD patients, strategies have been conceived to support gluten avoidance or detoxification. A key determinant of the intestinal fate of gluten and the physiological response to it is the intestinal microbiota, as has been revealed from experiments with differentially colonized mice [8] and from comparisons of microbiota from CD patients versus healthy individuals [9, 10]. In line with this, several microbiota-targeted technologies have been developed with the aim to ameliorate gluten-related disorders. These technologies can be categorized into: 1. Oral application of Lactobacillus spp. or Bifidobacterium spp. to correct dysbiosis associated with GFD or gluten-related disorders, 2. Oral application of Lactobacillus spp. or Bifidobacterium spp. as non-specific support for gluten-related disorders via undefined mechanisms., 3. Oral application of Lactobacillus spp. or Bifidobacterium spp. to support the degradation of gluten, 4. Oral application of peptide hydrolases to support the degradation of gluten (“glutenases”). Degradation of toxic/immunogenic gluten epitopes requires combined activities of peptidases PepN, PepO, PepI, PepX, PepP [11].

Recently, the taxonomic classification of several species of the genus Lactobacillus has been updated, according to Zheng J, Wittouck S, Salvetti E, Cmap Franz HMB, Harris P, Mattarelli PW, O'Toole B, Pot P, Vandamme J, Walter K, Watanabe S, Wuyts GE, Felis MG, Ganzle A and Lebeer S, 2020. A taxonomic note on the genus lactobacillus: description of 23 novel genera, emended description of the genus lactobacillus Beijerinck 1901, and Union of Lactobacillaceae and Leuconostocaceae. International Journal of Systematic and Evolutionary Microbiology. Hypertext transfer protocol. secure://doi.org/10.1099/ijsem.0.004107. Of particular relevance in the context of this invention are the following species:

| “Old” denomination | Updated denomination (since 2020) |
|---|---|
| Lactobacillus brevis | Levilactobacillus brevis |
| Lactobacillus casei | Lacticaseibacillus casei |
| Lactobacillus paracasei | Lacticaseibacillus paracasei |
| Lactobacillus plantarum | Lactiplantibacillus plantarum |
| Lactobacillus reuteri | Limosilactobacillus reuteri |
| Lactobacillus sanfranciscensis | Fructilactobacillus sanfranciscensis |

AU2015261774 AA claims the use of Lactobacillus casei (Lacticaseibacillus casei) IPLA12038 to prevent or treat CD. This strain has been described to degrade a certain amount of the 33-mer, but not any other important immunogenic peptide, within 12 hours and to possess the following enzymatic activities: PepN 8.03 mEU/mg; PepQ 9.5 mEU/mg; PepI 0.58 mEU/mg; PepX 3.19 mEU/mg. The strain does not survive acidic conditions (pH<3.0) and therefore does not offer a technical solution for gluten-related disorders.

WO2017139659 A1 claims cleavage of XPQ motifs and only two immunogenic peptides (33-mer and 26-mer) by subtilisins from Rothia species.

AU2008341708 AA claims the use of Bifidobacterium longum CECT 7347 in the treatment of gluten-related disorders; no reference is made to any peptidase or protease activities of this strain against gluten or critical epitopes therein.

WO17134240 A1 claims compositions containing the species Faecalibacterium prausnitzii, Butyricicoccus pullicaecorum, Roseburia inulinivorans, Roseburia hominis, Akkermansia muciniphila, Lactobacillus plantarum (Lactiplantibacillus plantarum) and Anaerostipes caccae for the treatment of CD.

US2017000830 AA claims compositions containing Lactococcus species and enzymes for amelioration of gluten sensitivity. Francavilla et al. reported improvement of irritable bowel syndrome (IBS)-like symptoms of CD patients on a GFD after application of a combination of five strains from the genera Lactobacillus and Bifidobacterium [12]. This treatment was associated with a shift in gut microbiota composition; effects of the strains on gluten digestion were however not reported. Meanwhile, the same group assessed in vitro peptidase activities of Lactobacillus strains, showing activities of up to 10 mU/mg for PepN, 10 mU/mg for PepI, 5 mU/mg for PEP, 25 mU/mg for PepQ for strains of the species Lactobacillus plantarum (Lactiplantibacillus plantarum), Lactobacillus bulgaricus, Lactobacillus rhamnosus, Lactobacillus paracasei (Lacticaseibacillus paracasei), and Lactobacillus casei (Lacticaseibacillus casei) [13]. Combined application of ten of these strains led to hydrolysis of gliadin epitopes listed in Table 1 after 24 hours of incubation. Survival of the strains under gastric and small intestinal conditions was not determined, the effectiveness of these strains on gluten digestion in the gastrointestinal tract of humans can therefore not be predicted.

Herran et al. isolated 27 bacterial strains belonging to the species L. salivarius, L. rhamnosus, L. reuteri (Limosilactobacillus reuteri), L. casei (Lacticaseibacillus casei), L. oris, L. gasseri, L. fermentum, L. crispatus, L. brevis (Levilactobacillus brevis), B. subtilis, B. amyloliquefaciens, B. pumilus, and B. licheniformis from the small intestine of humans that showed proteolytic activity against the 33-mer only after a very long incubation time of 24 hours and not against other peptides [14]. Similarly, weak activity against this epitope was found for other strains of human small intestinal origin, again including the species B. subtilis, B. pumilus, and B. licheniformis [15].

CA3069659A1 discloses a method for preparing gluten-free flour by compositions containing fungal enzymes and probiotic bacteria selected from the species Bacillus amyloliquefaciens, Lactobacillus brevis (Levilactobacillus brevis), Lactobacillus delbrueckii, Lactobacillus reuteri (Limosilactobacillus reuteri), and Lactobacillus helvetivus. Survival of these bacteria under gastric and small intestinal conditions was not determined, the effectiveness of these strains on gluten digestion in the gastrointestinal tract of humans can therefore not be predicted. Moreover, the fate of gluten digested by these compositions has not been disclosed; neither the appearance or disappearance of immunogenic peptides nor the immunogenicity of the digests have been assessed.

The commercial use of peptide hydrolases with the intention to detoxify gluten during food processing [16] and in humans [17] has been described. However, related products containing these enzymes have minimal evidence of efficacy [17].

Conclusively, the problems of gluten-related disorders remain unsolved, as all the above-mentioned applications have several problems and have not been successfully translated into the clinic. Applications 1 and 2 may merely provide an indirect benefit, as the underlying problem of gluten toxicity is not addressed directly. Application 3 has so far been limited to ex vivo digestion of gluten, assessment of solely protease activity against gluten/gliadin proteins, leading to only partial digestion, or assessment of digestion of only very few immunogenic gluten peptides after very long incubation times of 12 hours or longer.

The lack of successful clinical translation can be explained by poor survival of the combined microbial strains under realistic conditions in the gastrointestinal tract, and due to limited activity against gluten peptides in food matrices.

Microbial enzyme treatments for CD patients (Application 4) have the major limitation of poor proteolytic resistance, extent and duration of enzymatic activity during gastrointestinal transit [18]. Moreover, they are even considered as hazardous because they can only partially degrade gluten and thereby potentially release toxic epitopes [17].

It was aimed to overcome these limitations by developing an inventive and effective technology based on the following considerations:
Gluten/gliadin/glutenin degradation during human digestion is not beneficial per se, because incomplete degradation can lead to the formation of toxic and/or immunogenic peptides
Concerns have been expressed on the safety of currently available means to trigger gluten degradation in vivo, as these may do so only partially and can thereby induce or worsen gluten toxicity
Any attempt to trigger gluten degradation in vivo needs to make sure that such degradation is complete and leads to safe degradation products
Given the diversity of gluten-inherent peptide sequences with immunogenic potential, a combination of peptide hydrolases from different microbes is required to ensure complete degradation of all peptides
Such combination is preferably provided by a consortium of probiotic microorganisms that are metabolically active and synergize with each other in relevant parts of the gastrointestinal tract (i.e. the stomach and duodenum) to promote a safe, rapid, and complete digestion of gluten proteins from relevant food matrices to non-toxic, non-immunogenic small peptides or amino acids
We conceived that such a synergism can be achieved by combining acid- and bile-resistant strains with suitable protein/peptide substrate specificities and found that combinations of certain Lactobacillus sp., Bacillus sp., and in some cases also Pediococcus sp., including their cytoplasmic extracts, from specific ecological niches are particularly useful for this

In this invention we disclose the application of consortia comprised by Lactobacillus and Bacillus species, with optional addition of Pediococcus strains as a technology for use in the degradation of gluten in the gastrointestinal tract of humans and animals as well as during food production.

The means how such technology can bring a benefit to people in need thereof as well as to people willing to minimize their exposure to gluten for precautionary or other reasons is as follows:
(i) Safe clearance of intentionally or accidentally ingested gluten as a cure or complementing therapy for CD, WA, and NCGS patients. The possibility to return to a conventional, gluten-containing diet.
(ii) Safe clearance of intentionally or accidentally ingested gluten as a cure or complementing therapy for people with non-specific intestinal or extra-intestinal symptoms that may result from ingested gluten. The possibility to return to a conventional, gluten-containing diet.
(iii) Offering a solution for symptom-free people wanting to minimize their gluten exposure as an alternative to adhering to a GFD.

The present invention is directed to preparations comprising Lactobacillus and Bacillus species, with optional addition of Pediococcus strains. These new preparations promote the digestion of gluten to non-toxic and non-immunogenic peptides/amino acids in the human gastrointestinal tract and during the production of gluten-containing food stuffs.

Subject of the present invention is therefore a preparation comprising consortia of at least one probiotic strain selected from the genus Bacillus and at least one probiotic strain selected from the genus Lactobacillus, for use in safe and complete degradation of gluten.

The present invention is directed to a preparation comprising consortia of at least one bacterial strain selected from the genus Bacillus and at least one bacterial strain selected from the genus Lactobacillus, for use in the degradation of gluten to a gluten content of 20 ppm or less,
a) wherein said consortium of strains can degrade the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), the 33-mer peptide LQLQPFPQPQLPYPQPQLPYPQPQLPYPQPQPF (Seq-ID No 4).

In a preferred configuration, the consortium of strains can degrade the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), the 33-mer peptide LQLQPFPQPQLPYPQPQLPYPQPQLPYPQPQPF (Seq-ID No 4) by at least 1% or at least 10%, or at least 20% or at least 30% or at least 40%, or at least 50% or at least 60% or at least 70%, preferably by at least 80%, or at least 90%, preferably at least 95%, more preferably at least 98%.

The gluten content is determined via an ELISA assay, preferably by either determining hydrolysed gluten according to a AOAC (Association of Official Agricultural Chemists) International Official Method of Analysis (OMA) (Method No. AACCI 38-55.01) using R5 antibody-based sandwich and competitive ELISA (R5-ELISA) [22] or by determining residual gluten using a , ELISA Systems Gluten Residue Detection Kit (Windsor, Australia).

In a preferred configuration, the preparation is able to reduce the residual gluten by at least 85%, preferably at least 90%, more preferably at least 95% after 6 h, or by at least 95%, more preferably at least 98% after 16 h, or by at least 99% after 48 h. Alternatively, the preparation is able to reduce gluten fragments by at least 90% after 6 h, or by at least 95% after 16 h, or by at least 97% after 48 h.

In another preferred configuration, the preparation is able to reduce the residual gluten by at least 95%, preferably at least 97% after 6 h, or by at least 99% after 16 h. Alternatively, the preparation is able to reduce gluten fragments by at least 94% after 6 h, or by at least 97% after 16 h, or by 100% after 48 h.

A further preferred configuration is a preparation for use,
b) wherein said consortium of strains is capable of degrading gluten to a digest that does not cause an immunogenic or toxic response in the small intestine or small intestinal explant of a subject or animal affected by a gluten-related disorder, and/or
c) wherein all strains of the consortium survive (less than 2 log CFU loss) simulated gastric (pH 1.0-4.0) and intestinal (pH 5.4-6.8; 0.05-0.6% bile acids, or the concentration of bile acids present in humans under in vivo conditions) conditions, and/or
d) wherein members of the consortia have complementary PepP, PepO, PepX, PepI, and PepN activities with at least one peptidase activity equal to or more than 3 U/g (PepP), 5 U/g (PepO), 20 U/g (PepX), 17 U/g (PepI), 20 U/g (PepN).

Bacteria of the species Bacillus subtilis, Bacillus pumilus, Bacillus licheniformis and Bacillus megaterium, Bacillus amyloliquefaciens and Lactobacillus plantarum (Lactiplantibacillus plantarum), Lactobacillus paracasei (Lacticaseibacillus paracasei), Lactobacillus sanfranciscensis (Fructilactobacillus sanfranciscensis), Lactobacillus brevis (Levilactobacillus brevis), Lactobacillus reuteri (Limosilactobacillus reuteri), and in some cases also Pediococcus sp. were found to be especially suitable for this effect.

Therefore, in a preferred embodiment, the Bacillus strains are selected from Bacillus pumilus, Bacillus subtilis, Bacillus licheniformis, Bacillus megaterium, preferably selected from Bacillus pumilus DSM 33297, DSM 33355, DSM 33301, Bacillus subtilis DSM 33353, DSM 33298, Bacillus licheniformis DSM 33354, Bacillus megaterium DSM 33300, DSM 33356.

The Lactobacillus strains are selected from Lactobacillus plantarum (Lactiplantibacillus plantarum), Lactobacillus casei (Lacticaseibacillus casei), Lactobacillus paracasei (Lacticaseibacillus paracasei), Lactobacillus brevis (Levilactobacillus brevis), Lactobacillus sanfranciscensis (Fructilactobacillus sanfranciscensis), Lactobacillus reuteri (Limosilactobacillus reuteri), preferably selected from Lactobacillus plantarum (Lactiplantibacillus plantarum) DSM 33362, DSM 33363, DSM 33364, DSM 33366, DSM 33367, DSM 33368, DSM 33369, DSM 33370, Lactobacillus paracasei (Lacticaseibacillus paracasei) DSM 33373, DSM 33375, DSM 33376, Lactobacillus reuteri (Limosilactobacillus reuteri) DSM 33374, Lactobacillus brevis (Levilactobacillus brevis) DSM 33377, Lactobacillus sanfranciscensis (Fructilactobacillus sanfranciscensis) DSM 33378, DSM 33379.

In a preferred embodiment, the preparation for use according to the present invention comprises one or more of the following strains:

L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300, and Bacillus subtilis DSM 33353, or

L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298, DSM 33353, or

L. plantarum (Lactiplantibacillus plantarum) DSM 33366, DSM 33369; Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356, and Bacillus subtilis DSM 33298, or

L. plantarum (Lactiplantibacillus plantarum) DSM 33363 and DSM 33364, L. paracasei (Lacticaseibacillus paracasei) DSM 33373, Bacillus pumilus DSM 33297 and Bacillus megaterium DSM 33300, or

L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301.

Particularly preferred preparations for use according to the present invention comprise the following strains:

L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or

L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or

L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355.

The cells of the strains of the current invention may be present in the compositions of the current invention, as spores (which are dormant), as vegetative cells (which are growing), as transition state cells (which are transitioning from vegetative cells to spores, or reverse), as whole cell extracts or as enriched enzyme fractions or purified enzymes or as a combination of at least two of these types of cells or extracts/isolates.

The current invention is also related to compositions which comprise microbial strains into which protease genes isolated from the above-mentioned strains have been transferred by means of gene cloning and gene transferal procedures and the use of such genetically engineered strains as spores (if applicable), as vegetative cells, as transition state cells, as whole cell extracts or as enriched enzyme fractions or purified enzymes or as a combination of at least two of these types of cells or extracts/isolates.

In a preferred embodiment, the probiotic strain is present in a dormant form or as vegetative cells. In alternative embodiment, cytoplasmic extracts or cell-free supernatants or heat-killed biomass of the probiotic strains are used.

In a further preferred embodiment, the preparation further comprises one or more probiotic strains, preferably selected from Pediococcus sp., Weissella sp., more preferably Pediococcus pentosaceus DSM 33371.

In a further preferred embodiment, the preparation further comprises one or more of the following: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase, arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum, xylooligosaccharides, alginate.

The invention is also directed to preparations for use for treating or preventing gluten-related disorders, preferably selected from celiac disease, non-celiac gluten sensitivity, wheat allergy, and gluten-sensitive irritable bowel syndrome in a subject or animal in need thereof.

Moreover, the invention is directed to preparations for use for producing gluten-free foods, from gluten-containing cereals wheat, barley, rye, and oat, preferably containing less than 20 ppm gluten.

In a preferred embodiment, the preparation for use further comprises a substance, which acts as permeabilizer of the microbial cell membrane of members of Bacillus sp., Lactobacillus sp., Pediococcus sp., Weissella sp., preferably alginate.

In an alternative embodiment, one or more of the probiotic strains selected from Bacillus sp., Lactobacillus sp., Pediococcus sp. and Weissella sp are immobilized individually or as consortia. Immobilization can be realized on solid surfaces such as cellulose and chitosan, as entrapment within a porous matrix such as polysaccharide gels like alginates, k-carrageenan, agar, chitosan and polygalacturonic acid or other polymeric matrixes like gelatin, collagen and polyvinyl alcohol or by flocculation and microencapsulation or electrospraying technologies. Strains of the genera Lactobacillus, Bacillus, and Pediococcus were screened for survival under simulated gastrointestinal conditions and for PepN, PepO, PEP, PepX, PepQ activities. The following strains survived under simulated gastrointestinal conditions (less than 2 log reduction of CFU) and showed exceptionally high peptidase activities such as PepN, PepO, PEP, PepX, PepQ:
Lactobacillus plantarum (Lactiplantibacillus plantarum) DSM 33370, DSM 33362, DSM 33363, DSM 33364, DSM 33367, DSM 33366, DSM 33369, DSM 33368
Lactobacillus reuteri (Limosilactobacillus reuteri) DSM 33374
Lactobacillus brevis (Levilactobacillus brevis) DSM 33377
Lactobacillus paracasei (Lacticaseibacillus paracasei) DSM 33373, DSM 33375, DSM 33376
Lactobacillus sanfranciscensis (Fructilactobacillus sanfranciscensis) DSM 33378, DSM 33379
Bacillus subtilis DSM 33298, DSM 33353
Bacillus pumilus DSM 33301, DSM 33297, DSM 33355
Bacillus megaterium DSM 33300, DSM 33356
Bacillus licheniformis DSM 33354
Pediococcus pentosaceus DSM 33371

Thus Bacillus/Lactobacillus/Pediococcus strains that are preferably used for preparations according to the present invention are selected from the following groups:

The Bacillus subtilis strains as deposited under DSM 33298, DSM 33353 at the DSMZ; the Bacillus pumilus strains as deposited under DSM 33301, DSM 33297, DSM 33355 at the DSMZ; the Bacillus megaterium strains as deposited under DSM 33300, DSM 33356 at the DSMZ; the Bacillus licheniformis strain as deposited under DSM 33354 at the DSMZ; the Pediococcus pentosaceus strain as deposited under DSM 33371 at the DSMZ; the Lactobacillus sanfranciscensis (Fructilactobacillus sanfranciscensis) strains as deposited under DSM 33378, DSM 33379 at the DSMZ; the Lactobacillus plantarum (Lactiplantibacillus plantarum) strains as deposited under DSM 33370, DSM 33362, DSM 33363, DSM 33364, DSM 33367, DSM 33366, DSM 33369, DSM 33368 at the DSMZ; the Lactobacillus reuteri (Limosilactobacillus reuteri) strain as deposited under DSM 33374 at the DSMZ; the Lactobacillus brevis (Levilactobacillus brevis) strain as deposited under DSM 33377 at the DSMZ; the Lactobacillus paracasei (Lacticaseibacillus paracasei) strains as deposited under DSM 33373, DSM 33375, DSM 33376 at the DSMZ.

When strains L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300, Bacillus subtilis DSM 33353 were combined (combination 1), all the four tested gluten epitopes listed in Table 1 were completely degraded within 12 hours.

Likewise, combination 2, which comprised L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298, DSM 33353,

and combination 3, which comprised L. plantarum (Lactiplantibacillus plantarum) DSM 33366 and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356, Bacillus subtilis DSM 33298 led to complete degradation of gluten epitopes listed in Table 1 within 12 hours.

Furthermore, a combination of Lactobacillus plantarum (Lactiplantibacillus plantarum) DSM 33363 and DSM 33364, Lactobacillus paracasei (Lacticaseibacillus paracasei) DSM 33373, and Bacillus pumilus DSM 33297 and Bacillus megaterium DSM 33300 were similarly effective as strain combinations 1-3.

Furthermore, a combination of Lactobacillus plantarum (Lactiplantibacillus plantarum) DSM 33363 and DSM 33364, Lactobacillus paracasei (Lacticaseibacillus paracasei) DSM 33373, Lactobacillus reuteri (Limosilactobacillus reuteri) DSM 33374, Bacillus pumilus DSM 33297 and DSM 33355, and Bacillus megaterium DSM 33300 were similarly effective as strain combinations 1-3.

Therefore, a preferred configuration of the present invention is directed to a preparation comprising strain combinations selected from the following:
L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300 and Bacillus subtilis DSM 33353, or
L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298 and DSM 33353, or
L. plantarum (Lactiplantibacillus plantarum) DSM 33366 and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356 and Bacillus subtilis DSM 33298, or
Lactobacillus plantarum (Lactiplantibacillus plantarum) DSM 33363 and DSM 33364, Lactobacillus paracasei (Lacticaseibacillus paracasei) DSM 33373, and Bacillus pumilus DSM 33297 and Bacillus megaterium DSM 33300, or
Lactobacillus plantarum (Lactiplantibacillus plantarum) DSM 33363 and DSM 33364, Lactobacillus paracasei (Lacticaseibacillus paracasei) DSM 33373, Lactobacillus reuteri (Limosilactobacillus reuteri) DSM 33374, Bacillus pumilus DSM 33297 and DSM 33355, and Bacillus megaterium DSM 33300.

Numerous other combinations among the above listed strains of Bacillus sp., Lactobacillus sp., and Pediococcus sp. showed comparable performance to those already indicated.

More specifically, preferred configurations of this invention comprise strain combinations that in sum provide high enzymatic PepI, PepN, PepX, PepO, and PepP activity; consequently, such combinations contain at least one strain of each of the following groups 1-5, wherein group members have particularly high enzymatic activity for PepI (group 1), PepN (group 2), PepX (group 3), PepO (group 4), PepP (group 5).

Another subject of the present invention is therefore a preparation comprising at least one strain of each of the following groups 1-5:
Group 1: Lactobacillus paracasei (Lacticaseibacillus paracasei) DSM 33373, Lactobacillus paracasei (Lacticaseibacillus paracasei) DSM 33375,
Group 2: Bacillus subtilis DSM 33298, Bacillus pumilus DSM 33297, Bacillus licheniformis DSM 33354, Bacillus megaterium DSM 33356, Pediococcus pentosaceus DSM 33371, Lactobacillus plantarum (Lactiplantibacillus plantarum) DSM 33370, Lactobacillus brevis (Levilactobacillus brevis) DSM 33377, Lactobacillus paracasei (Lacticaseibacillus paracasei) DSM 33376, Lactobacillus paracasei (Lacticaseibacillus paracasei) DSM 33375, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33363, Lactobacillus paracasei (Lacticaseibacillus paracasei) DSM 33373, Lactobacillus plantarum (Lactiplantibacillus plantarum) DSM 33362,
Group 3: Lactobacillus sanfranciscensis (Fructilactobacillus sanfranciscensis) DSM 33378, Lactobacillus sanfranciscensis (Fructilactobacillus sanfranciscensis) DSM 33379, Pediococcus pentosaceus DSM 33371, Lactobacillus plantarum (Lactiplantibacillus plantarum) DSM 33370, Lactobacillus plantarum (Lactiplantibacillus plantarum) DSM 33369, Lactobacillus reuteri (Limosilactobacillus reuteri) DSM 33374, Lactobacillus plantarum (Lactiplantibacillus plantarum) DSM 33363, Lactobacillus paracasei (Lacticaseibacillus paracasei) DSM 33373,
Group 4: Bacillus subtilis DSM 33353, Bacillus pumilus DSM 33355, Bacillus pumilus DSM 33301,
Group 5: Bacillus megaterium DSM 33300, Lactobacillus sanfranciscensis (Fructilactobacillus sanfranciscensis) DSM 33378, Pediococcus pentosaceus DSM 33371, Lactobacillus brevis (Levilactobacillus brevis) DSM 33377, Lactobacillus plantarum (Lactiplantibacillus plantarum) DSM 33368, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33366, Lactobacillus plantarum (Lactiplantibacillus plantarum) DSM 33364, Lactobacillus paracasei (Lacticaseibacillus paracasei) DSM 33373.

In a preferred configuration, the preparation comprises at least three different strains, preferably at least four different strains, more preferably at least five different strains.

It is particularly preferred, when the preparation comprises the following strains:
L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300 and Bacillus subtilis DSM 33353, or
L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298 and DSM 33353, or
L. plantarum (Lactiplantibacillus plantarum) DSM 33366 and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356 and Bacillus subtilis DSM 33298, or
L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or
L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355.
L. plantarum (Lactiplantibacillus plantarum) DSM 33370, DSM 33363 and DSM 33364, L. paracasei (Lacticaseibacillus paracasei) DSM 33373 L. brevis (Levilactobacillus brevis) DSM 33377, Bacillus pumilus DSM 33297, DSM 33355, DSM 33301, or
L. plantarum (Lactiplantibacillus plantarum) DSM 33362 and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300, or
L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353, or
L. brevis (Levilactobacillus brevis) DSM 33377, Pediococcus pentosaceus DSM 33371, L. plantarum (Lactiplantibacillus plantarum) DSM 33369, Bacillus pumilus DSM 33297 and Bacillus megaterium DSM 33300, or
L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354, or
L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353, or
L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354, or
L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356, or
L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353, or
L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355, or
L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297, or
L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354, or
L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, B. subtilis DSM 33353.

In a further preferred configuration, the preparation comprises the following strains:
L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or
L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355.

Another preferred configuration of the present invention are formulations to be used in the preparation of food stuffs from cereals by e.g. fermentation and baking processes.

Therefore, the invention is also related to a food or pet food supplement or food or pet food product, comprising a preparation according to the present invention.

One subject of the present invention is the use of a preparation according to the present invention as a food supplement or its use in foodstuffs. Preferred foodstuffs according to the invention are chocolate products, gummies, mueslis, muesli bars, and dairy products.

A further subject of the current invention is also the use of a preparation of the current invention as a synbiotic ingredient in food or feed products.

A further subject of the present invention is a foodstuff composition containing a preparation according to the present invention and at least one further food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof.

The foodstuff or feedstuff composition according to the present invention does also include dietary supplements, e. g. in the form of a pill, capsule, tablet, powder or liquid.

A further subject of the current invention is a pharmaceutical composition containing a preparation according to the present invention and a pharmaceutically acceptable carrier.

The preparations according to the present invention, when administered to human beings or animals, preferably improve the health status, in particular gut health, cardiovascular health, mental health, or immune health of a human being.

An advantageous configuration according to the present invention is a composition for improving the health status of a human being or an animal by one or more of the following:
decreasing the amount of toxic gluten epitopes, preferably those listed in Table 1.
decreasing the amount of initial gluten to less than 20 ppm.

### Items

1. Preparation comprising consortia of at least one bacterial strain selected from the genus Bacillus and at least one bacterial strain selected from the genus Lactobacillus, for use in the degradation of gluten.
2. Preparation comprising consortia of at least one bacterial strain selected from the genus Bacillus and at least one bacterial strain selected from the genus Lactobacillus, for use in the degradation of gluten to a gluten content of 20 ppm or less.
3. Preparation for use according to item 1 or 2,
   a) wherein said consortium of strains can degrade the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), the 33-mer peptide LQLQPFPQPQLPYPQPQLPYPQPQLPYPQPQPF (Seq-ID No 4).
4. Preparation for use according to any preceding item, wherein the gluten content is determined via an ELISA assay, preferably an antibody-based sandwich or competitive assay.
5. Preparation for use according to any preceding item, wherein the preparation is able to reduce the residual gluten by at least 85%, preferably at least 90%, more preferably at least 95% after 6 h, or by at least 95%, more preferably at least 98% after 16 h, or by at least 99% after 48 h.
6. Preparation for use according to any preceding item, wherein the preparation is able to reduce gluten fragments by at least 90% after 6 h, or by at least 95% after 16 h, or by at least 97% after 48 h.
7. Preparation for use according to any preceding item, wherein the preparation is able to reduce the residual gluten by at least 95%, preferably at least 97% after 6 h, or by at least 99% after 16 h.
8. Preparation for use according to any preceding item, wherein the preparation is able to reduce gluten fragments by at least 94% after 6 h, or by at least 97% after 16 h, or by 100% after 48 h.
9. Preparation for use according to any preceding item, wherein the consortium of strains can degrade the 12-mer peptide QLQPFPQPQLPY (Seq-ID No 1), the 14-mer peptide 40 PQPQLPYPQPQSFP (Seq-ID No 2), the 20-mer peptide QQLPQPQQPQQSFPQQQRPF (Seq-ID No 3), the 33-mer peptide LQLQPFPQPQLPYPQPQLPYPQPQLPYPQPQPF (Seq-ID No 4) by at least 80%, or at least 90%, preferably at least 95%, more preferably at least 98%.
10. Preparation for use according to any preceding item,
   b) wherein said consortium of strains is capable of degrading gluten to a digest that does not cause an immunogenic or toxic response in the small intestine or small intestinal explant of a subject or animal affected by a gluten-related disorder, and/or
   c) wherein all strains of the consortium survive (less than 2 log CFU loss) simulated gastric (pH 1.0-4.0) and intestinal (pH 5.4-6.8; 0.05-0.6% bile acids, or the concentration of bile acids present in humans under in vivo conditions) conditions, and/or
   d) wherein members of the consortia have complementary PepP, PepO, PepX, PepI, and PepN activities with at least one peptidase activity equal to or more than 3 U/g (PepP), 5 U/g (PepO), 20 U/g (PepX), 17 U/g (PepI), 20 U/g (PepN).
11. Preparation for use according to any preceding item, wherein the Bacillus strains are selected from Bacillus pumilus, Bacillus subtilis, Bacillus licheniformis, Bacillus megaterium, preferably selected from Bacillus pumilus DSM 33297, DSM 33355, DSM 33301, Bacillus subtilis DSM 33353, DSM 33298, Bacillus licheniformis DSM 33354, Bacillus megaterium DSM 33300, DSM 33356.
12. Preparation for use according to any preceding item, wherein the Lactobacillus strains are selected from Lactobacillus plantarum (Lactiplantibacillus plantarum), Lactobacillus casei (Lacticaseibacillus casei), Lactobacillus paracasei (Lacticaseibacillus paracasei), Lactobacillus brevis (Levilactobacillus brevis), Lactobacillus sanfranciscensis (Fructilactobacillus sanfranciscensis), Lactobacillus reuteri (Limosilactobacillus reuteri), preferably selected from Lactobacillus plantarum (Lactiplantibacillus plantarum) DSM 33362, DSM 33363, DSM 33364, DSM 33366, DSM 33367, DSM 33368, DSM 33369, DSM 33370, Lactobacillus paracasei (Lacticaseibacillus paracasei) DSM 33373, DSM 33375, DSM 33376, Lactobacillus reuteri (Limosilactobacillus reuteri) DSM 33374, Lactobacillus brevis (Levilactobacillus brevis) DSM 33377, Lactobacillus sanfranciscensis (Fructilactobacillus sanfranciscensis) DSM 33378, DSM 33379.
13. Preparation for use according to any preceding item, wherein the probiotic strains are present in a dormant form or as vegetative cells.
14. Preparation for use according to any preceding item, wherein cytoplasmic extracts or cell-free supernatants or heat-killed biomass of the probiotic strains are used.
15. Preparation for use according to any preceding item, wherein the preparation further comprises one or more probiotic strains, preferably selected from Pediococcus sp., Weissella sp., more preferably Pediococcus pentosaceus DSM 33371.
16. Preparation for use according to any preceding item, wherein the preparation further comprises one or more of the following: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase, arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum, xylooligosaccharides, alginate.
17. Preparation for use according to any preceding item for treating or preventing gluten-related disorders, preferably selected from celiac disease, non-celiac gluten sensitivity, wheat allergy, and gluten-sensitive irritable bowel syndrome in a subject or animal in need thereof.
18. Preparation for use according to any preceding item for producing gluten-free foods, from gluten-containing cereals wheat, barley, rye, and oat, preferably containing less than 20 ppm gluten.
19. Preparation for use according to any preceding item, further comprising a substance, which acts as permeabilizer of the microbial cell membrane of members of Bacillus sp., Lactobacillus sp., Pediococcus sp., Weissella sp., preferably alginate.
20. Preparation for use according to any preceding item where one or more of the probiotic strains selected from Bacillus sp., Lactobacillus sp., Pediococcus sp. and Weissella sp are immobilized individually or as consortia.
21. Preparation comprising at least one strain of each of the following groups 1-5:
   Group 1: Lactobacillus paracasei (Lacticaseibacillus paracasei) DSM 33373, Lactobacillus paracasei (Lacticaseibacillus paracasei) DSM 33375,
   Group 2: Bacillus subtilis DSM 33298, Bacillus pumilus DSM 33297, Bacillus licheniformis DSM 33354, Bacillus megaterium DSM 33356, Pediococcus pentosaceus DSM 33371, Lactobacillus plantarum (Lactiplantibacillus plantarum) DSM 33370, Lactobacillus brevis (Levilactobacillus brevis) DSM 33377, Lactobacillus paracasei (Lacticaseibacillus paracasei) DSM 33376, Lactobacillus paracasei (Lacticaseibacillus paracasei) DSM 33375, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33363, Lactobacillus paracasei (Lacticaseibacillus paracasei) DSM 33373, Lactobacillus plantarum (Lactiplantibacillus plantarum) DSM 33362,
   Group 3: Lactobacillus sanfranciscensis (Fructilactobacillus sanfranciscensis) DSM 33378, Lactobacillus sanfranciscensis (Fructilactobacillus sanfranciscensis) DSM 33379, Pediococcus pentosaceus DSM 33371, Lactobacillus plantarum (Lactiplantibacillus plantarum) DSM 33370, Lactobacillus plantarum (Lactiplantibacillus plantarum) DSM 33369, Lactobacillus reuteri (Limosilactobacillus reuteri) DSM 33374, Lactobacillus plantarum (Lactiplantibacillus plantarum) DSM 33363, Lactobacillus paracasei (Lacticaseibacillus paracasei) DSM 33373,
   Group 4: Bacillus subtilis DSM 33353, Bacillus pumilus DSM 33355, Bacillus pumilus DSM 33301,
   Group 5: Bacillus megaterium DSM 33300, Lactobacillus sanfranciscensis (Fructilactobacillus sanfranciscensis) DSM 33378, Pediococcus pentosaceus DSM 33371, Lactobacillus brevis (Levilactobacillus brevis) DSM 33377, Lactobacillus plantarum (Lactiplantibacillus plantarum) DSM 33368, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33366, Lactobacillus plantarum (Lactiplantibacillus plantarum) DSM 33364, Lactobacillus paracasei (Lacticaseibacillus paracasei) DSM 33373.
22. Preparation according to item 21, comprising at least three different strains, preferably at least four different strains, more preferably at least five different strains.
23. Preparation according to item 21 or 22, comprising the following strains:
   L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300 and Bacillus subtilis DSM 33353, or
   L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298 and DSM 33353, or
   L. plantarum (Lactiplantibacillus plantarum) DSM 33366 and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356 and Bacillus subtilis DSM 33298, or
   L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or
   L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
   L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355, or
   L. plantarum (Lactiplantibacillus plantarum) DSM 33370, DSM 33363 and DSM 33364, L. paracasei (Lacticaseibacillus paracasei) DSM 33373 L. brevis (Levilactobacillus brevis) DSM 33377, Bacillus pumilus DSM 33297, DSM 33355, DSM 33301, or
   L. plantarum (Lactiplantibacillus plantarum) DSM 33362 and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300, or
   L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353, or
   L. brevis (Levilactobacillus brevis) DSM 33377, Pediococcus pentosaceus DSM 33371, L. plantarum (Lactiplantibacillus plantarum) DSM 33369, Bacillus pumilus DSM 33297 and Bacillus megaterium DSM 33300, or
   L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354, or
   L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353, or
   L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354, or
   L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356, or
   L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353, or
   L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355, or
   L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297, or
   L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354, or
   L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, B. subtilis DSM 33353.
24. Preparation according to any item 21 to 23, comprising the following strains:
   L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or
   L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
   L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355.
25. Preparation according to any item 21 to 24, wherein the probiotic strains are present in a dormant form or as vegetative cells.
26. Preparation according to any item 21 to 25, wherein cytoplasmic extracts or cell-free supernatants or enriched enzyme fractions or purified enzymes or heat-killed biomass of the probiotic strains are used.
27. Preparation according to any item 21 to 26, wherein the preparation further comprises one or more probiotic strains, preferably selected from Pediococcus sp., Weissella sp., more preferably Pediococcus pentosaceus DSM 33371.
28. Preparation according to any item 21 to 27, wherein the preparation further comprises one or more of the following: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase, arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum, xylooligosaccharides, alginate.
40 29. A food or pet food supplement or food or pet food product or pharmaceutical product, comprising a preparation according to any one of items 21-28.

### WORKING EXAMPLES

### Example 1. Probiotic Microorganisms Resistant to Gastrointestinal Conditions

Simulated gastric and intestinal fluids were used as described by Fernández et al. [19]. Stationary-phase-grown cells were harvested at 8000 g for 10 min, washed with physiologic solution, and suspended in 50 ml of simulated gastric juice (cell density of 10 log CFU/ml), which contains NaCl (125 mM/I), KCl (7 mM/I), NaHCO3 (45 mM/I), and pepsin (3 g/I) (Sigma-Aldrich CO., St. Louis, Mo., USA) [20]. The final pH was adjusted to 2.0, 3.0, and 8.0. The value of pH 8.0 was used to investigate the influence of the components of the simulated gastric juice, apart from the effect of low pH. The suspension was incubated at 37° C. under anaerobic conditions and agitation to simulate peristalsis. Aliquots of this suspension were taken at 0, 90, and 180 min, and viable count was determined. The effect of gastric digestion was also determined by suspending cells in reconstituted skimmed milk (RSM) (11% solids, w/v) before inoculation of simulated gastric juice at pH 2.0. The final pH after the addition of RSM was ca. 3.0. This condition was assayed to simulate the effect of the food matrix during gastric transit [20]. After 180 min of gastric digestion, cells were harvested and suspended in simulated intestinal fluid, which contains 0.1% (w/v) pancreatin and 0.15% (w/v) Oxgall bile salt (Sigma-Aldrich Co.) at pH 8.0. The suspension was incubated at 37° C. under agitation and aliquots were taken at 0, 90, and 180 min [21]. 119 out of <400 tested strains showed a decrease of less than 2 log of initial 1×10¹⁰ CFU/ml and defined as resistant to simulated gastrointestinal conditions.

### Example 2. Protease and Peptidase Activities of Single Strains Resistant to Gastrointestinal Conditions

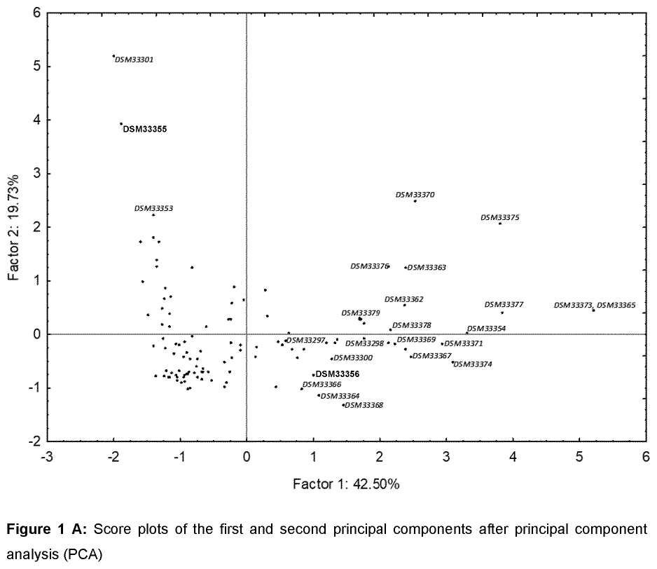

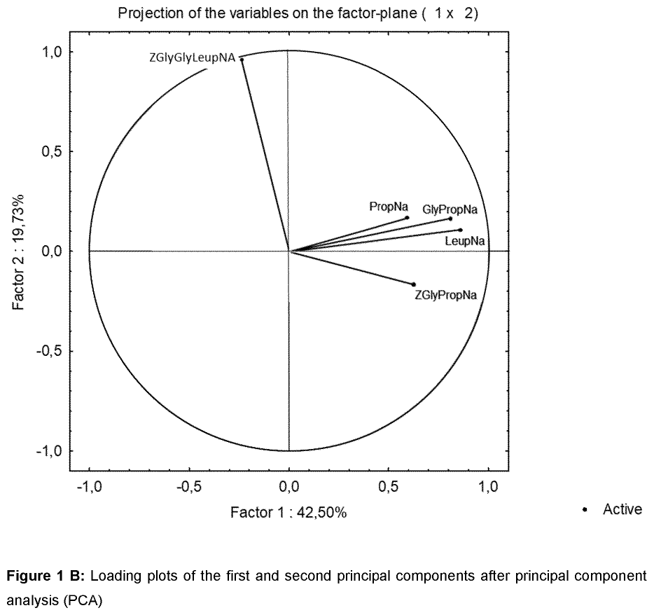

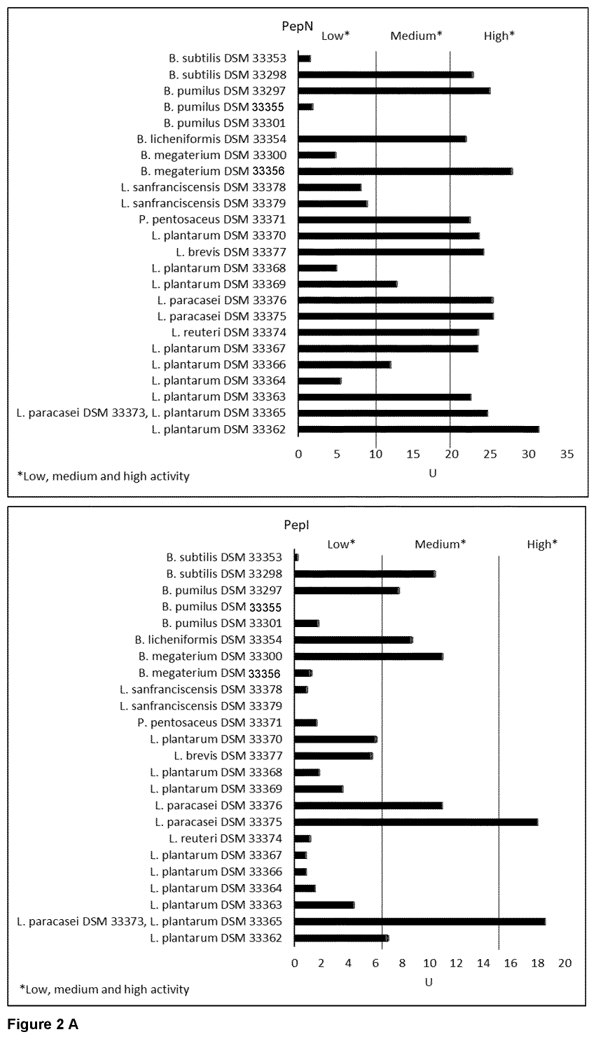

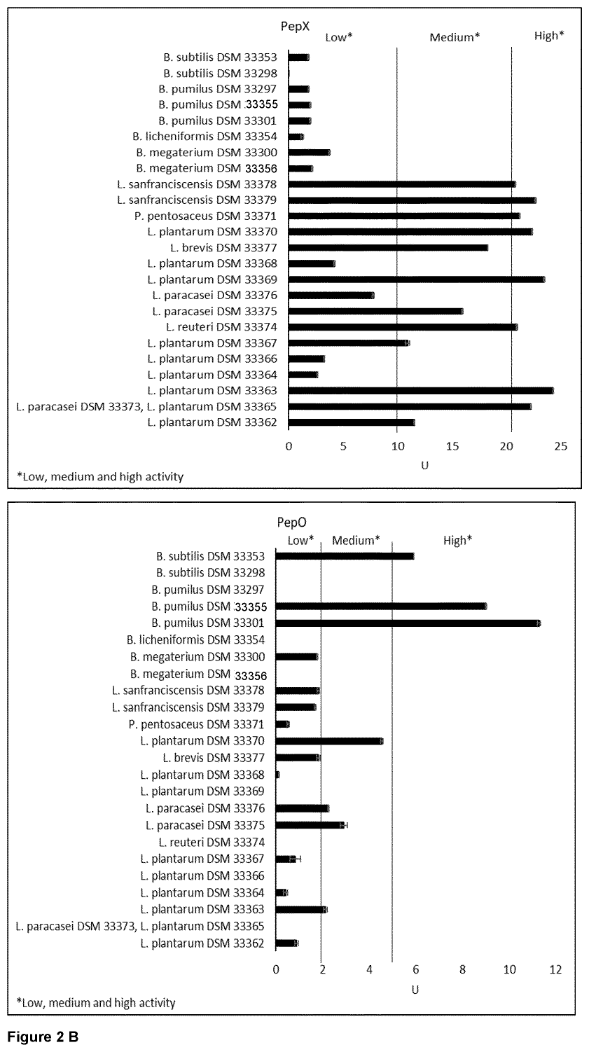

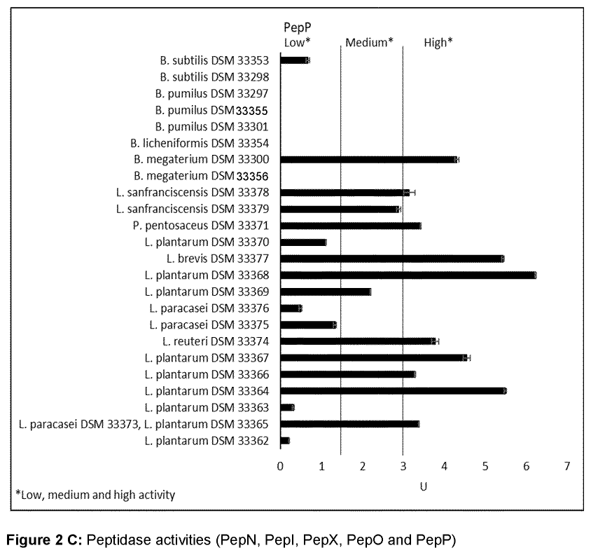

All 119 strains (Lactobacillus sp., 63 strains; Weissella sp., 3 strains; Pediococcus sp., 1 strain; and Bacillus sp., 51 strains) showing resistance to simulated gastrointestinal conditions were tested for their peptidase and proteinase activities towards synthetic substrates. To assay the peptidase activities, cultures of each strain from the late exponential phase of growth (ca. 9.0 log CFU/ml) were used. Aliquots (0.3 g [dry weight]) of washed cell pellets were re-suspended in 50 mM Tris-HCl (pH 7.0), incubated at 30° C. for 30 min, and centrifuged at 13,000×g for 10 min to remove enzymes loosely associated to the cell wall. The cytoplasmic extract was prepared by incubating bacterial suspensions with lysozyme in 50 mM Tris-HCl (pH 7.5) buffer containing 24% sucrose at 37° C. for 60 min, under stirring conditions (ca. 160 rpm). Spheroblasts were resuspended in isotonic buffer and sonicated for 40 sat 16 A/s (Sony Prep model 150; Sanyo, United Kingdom). The extracts were concentrated 10-fold by freeze-drying, re-suspended in 5 mM Tris-HCl (pH 7.0), and dialyzed for 24 h at 4° C. General aminopeptidase type N (PepN), proline iminopeptidase (PepI), X-prolyl dipeptidyl aminopeptidase (PepX) endopeptidase (PepO) and prolyl endopeptidase (PepP) activities of the cytoplasmic extracts of lactobacilli were measured by using Leu-p-nitroanilides (p-NA), Pro-p-NA, Gly-Pro-p-NA, Z-Gly-Gly-Leu-p-NA and Z-Gly-Pro-4-nitroanilide substrates (Sigma Chemical Co), respectively. The assay mixture contained 900 μl of 2.0 mM substrate in 0.05 M potassium phosphate buffer, pH 7.0, and 100 μl of cytoplasmic extract. The mixture was incubated at 37° C. for 180 min, and the absorbance was measured at 410 nm. The data were compared to standard curves set up by using p-nitroaniline. One unit of activity was defined as the amount of enzyme required to liberate 1 μmol of p-nitroaniline for min under the assay conditions. Based on Principal Component Analysis (PCA) data from the above peptidase activities, some strains clearly separated from the other ones (FIG. 1). FIG. 2 reports the strains showing very high peptidase activities (at least for one peptidase activity). PepN activity ranged from 0.0 (U002-C04; U541-C05; U776-C02; DSM 33301; U021-C01; DSM32540; U567-C04) to 31.400±0.09 U (DSM 33362) (median value 3.08). The strains with low peptidase activity (with the internal numbers/or deposited at the DSMZ: U002-C04; U541-C05; U776-C02; DSM 33301; U021-C01; DSM32540; U567-C04) were not further evaluated. The other most active strains were DSM 33367, DSM 33374, DSM 33370, DSM 33371, DSM 33377, DSM 33373, Bacillus pumilus DSM 33297, Bacillus subtilis DSM 33298, DSM 33376, DSM 33375, DSM 33363, Bacillus licheniformis DSM 33354, and Bacillus megaterium DSM 33356 (FIG. 1, FIG. 2). The median value of PepI was of 1.66. The most active strains (PepI activity>18 U) were DSM 33375, DSM 33373. PepX activity ranged from 0.0 to ca. 24 U. The most active strains were DSM 33379, DSM 33371, DSM 33370, DSM 33369, DSM 33374, DSM 33373, and DSM 33363 (FIG. 1 and FIG. 2) (median value of 1.81). The median value of PepO was of 0.54. The most active strains (PepO activity>5 U) were DSM 33353, DSM 33355, and DSM 33301. PepP activity ranged from 0.0 to 6.23 U (DSM 33368) (median value 0.22). The other most active strains (PepP activity>3 U) were Bacillus megaterium DSM 33300, DSM 33378, DSM 33371, DSM 33377, DSM 33367, DSM 33374, 20 DSM 33366, DSM 33373, and DSM 33364.

FIG. 1 shows the score (A) and loading (B) plots of the first and second principal components after principal component analysis (PCA) based on the general aminopeptidase type N (PepN), proline iminopeptidase (PepI), X-prolyl dipeptidyl aminopeptidase (PepX), endopeptidase (PepO) and prolyl endopeptidase (PepP) activities of the cytoplasmic extracts of the 119 Bacillus, Lactobacillus, Pediococcus, and Weissella strains. PepN, PepI, PepX, PepP were measured by using Leu-p-nitroanilides (p-NA), Pro-p-NA, Gly-Pro-p-NA, Z-Gly-Gly-Leu-p-NA and Z-Gly-Pro-4-nitroanilide substrates, respectively. Strains showing very high peptidase activities (at least for one peptidase) were reported in red.

FIG. 2 shows peptidase activities (PepN, PepI, PepX, PepO and PepP) of selected single Bacillus (B.), Lactobacillus (L.) and Pediococcus (P.) strains. One unit (U) of activity was defined as the amount of enzyme required to liberate 1 μmol of p-nitroanilide per min under the assay conditions.

### Example 3. Peptidase Activities of Mixture of Strains Against Immunogenic Epitopes

Bacillus, Lactobacillus, and Pediococcus strains showing very high peptidase activities (at least for one peptidase) were assessed as mixed strains to combine intense and complementary enzyme activities. Various mixtures were used to assay their capacity to in vitro degrade immunogenic epitopes responsible for gluten intolerance.

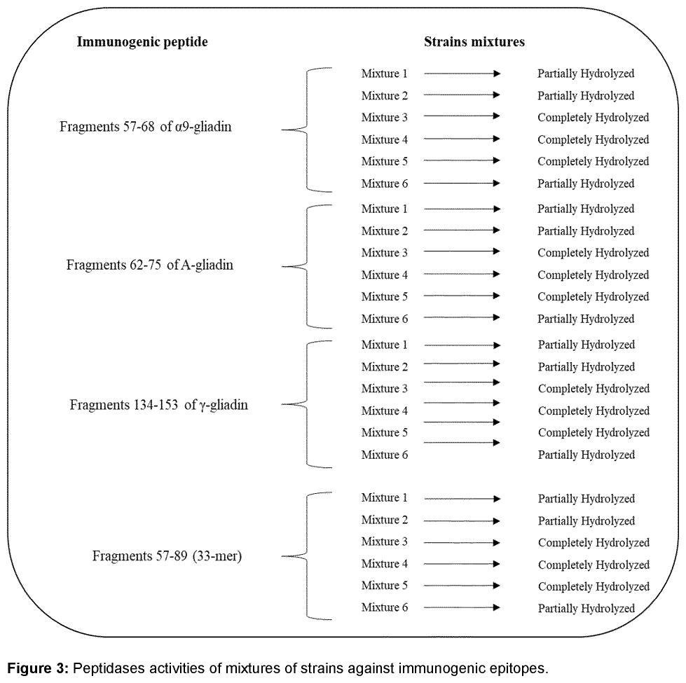

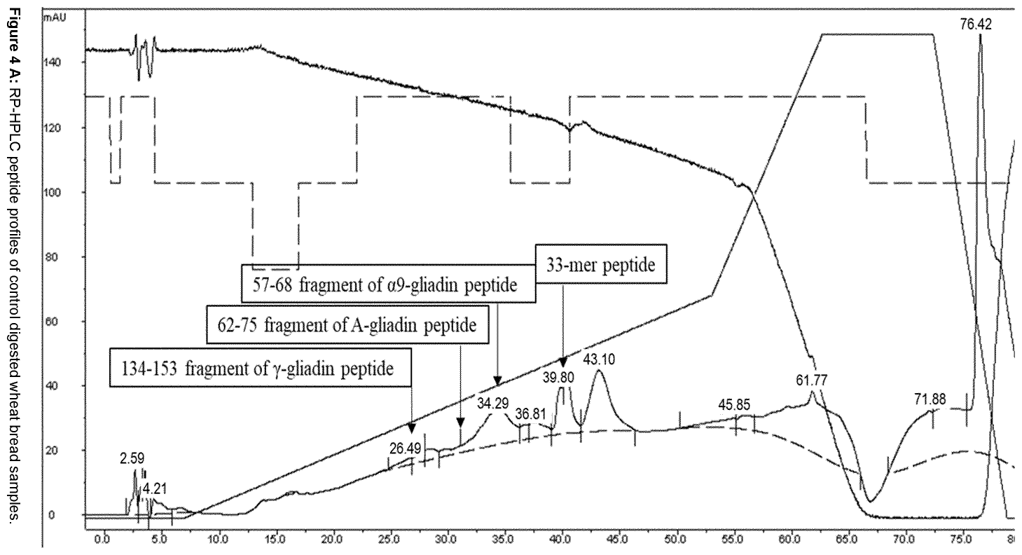

The hydrolysis of peptides was carried out using combinations of cytoplasmic extracts of previously selected bacteria strains. Immunogenic epitopes corresponding to fragments 57-68 (Q-L-Q-P-F-P-Q-P-Q-L-P-Y) of α9-gliadin, 62-75 (P-Q-P-Q-L-P-Y-P-Q-P-Q-S-F-P) of A-gliadin, 134-153 (Q-Q-L-P-Q-P-Q-Q-P-Q-Q-S-F-P-Q-Q-Q-R-P-F) of γ-gliadin, and 57-89 (L-Q-L-Q-P-F-P-Q-P-Q-L-P-Y-P-Q-P-Q-L-P-Y-P-Q-P-Q-L-P-Y-P-Q-P-Q-P-F) (33-mer) of α2-gliadin were chemically synthesized and used at an initial concentration of 1 mM. Hydrolysis was monitored by RP-HPLC. Single peaks from RP-HPLC were analysed by nano-ESI tandem mass spectrometry (nano-ESI-MS/MS). The mixtures of strains that showed the best hydrolysis of synthetic immunogenic epitopes were numbers 3, 4 and 5 (FIG. 3), which fully hydrolysed all toxic peptides (90% hydrolysis or more). FIG. 3 shows peptidase activities of mixtures of strains against immunogenic epitopes.

Strain mixtures were as follows:
1. L. plantarum (Lactiplantibacillus plantarum) DSM 33362, DSM 33363, DSM 33364, DSM 33366; L. sanfranciscensis (Fructilactobacillus sanfranciscensis) DSM 33379; Bacillus pumilus DSM 33297, DSM 33355, Bacillus licheniformis DSM 33354, Bacillus megaterium DSM 33300, Bacillus subtilis DSM 33353.
2. L. paracasei (Lacticaseibacillus paracasei) DSM 33375, DSM 33376; L. plantarum (Lactiplantibacillus plantarum) DSM 33369, DSM 33368; L. sanfranciscensis (Fructilactobacillus sanfranciscensis) DSM 33378; Bacillus licheniformis DSM 33354, Bacillus megaterium DSM 33300, DSM 33356, Bacillus pumilus DSM 33297, DSM 33301.
3. L. plantarum (Lactiplantibacillus plantarum) DSM 33370, DSM 33363, DSM 33364; Lactobacillus paracasei (Lacticaseibacillus paracasei) DSM 33373, L. brevis (Levilactobacillus brevis) DSM 33377; Bacillus pumilus DSM 33297, DSM 33355, Bacillus licheniformis DSM 33354, Bacillus megaterium DSM 33300, Bacillus subtilis DSM 33353.
4. L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, and Bacillus subtilis DSM 33298, DSM 33353.
5. L. plantarum (Lactiplantibacillus plantarum) DSM 33366, DSM 33369, Lactobacillus reuteri (Limosilactobacillus reuteri) DSM 33374; L. paracasei (Lacticaseibacillus paracasei) DSM 33376; Pediococcus pentosaceus DSM 33371, L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356, Bacillus subtilis DSM 33298.
6. L. plantarum (Lactiplantibacillus plantarum) DSM 33370, DSM 33367, Lactobacillus reuteri (Limosilactobacillus reuteri) DSM 33374; L. brevis (Levilactobacillus brevis) DSM 33377; Bacillus pumilus DSM 33301, Bacillus megaterium DSM 33300, DSM 33356, Bacillus subtilis DSM 33298.

### Example 4. Degradation of Gluten Under Simulated Gastrointestinal Conditions by Different Consortia

The gluten degradation under simulated gastrointestinal digestion was assessed. With the intention to develop a feasible technical solution for full degradation of gluten in vivo, we searched for minimal combinations containing as few strains as possible and as many as needed.

Using mixtures 1-6 of Example 3 as a starting point, the following consortia, selected from a total of 22 strains (Lactobacillus plantarum (Lactiplantibacillus plantarum) DSM 33370, DSM 33362, DSM 33363, DSM 33364, DSM 33366, DSM 33368, DSM 33369 and DSM 33367; Lactobacillus reuteri (Limosilactobacillus reuteri) DSM 33374; Lactobacillus paracasei (Lacticaseibacillus paracasei) DSM 33376, Lactobacillus paracasei (Lacticaseibacillus paracasei) DSM 33373, DSM 33375; Lactobacillus brevis (Levilactobacillus brevis) DSM 33377, Pediococcus pentosaceus DSM 33371; Bacillus pumilus DSM 33297, DSM 33355, DSM 33301, DSM 33355, Bacillus licheniformis DSM 33354, Bacillus megaterium DSM 33300, DSM 33356, and Bacillus subtilis DSM 33298, DSM 33353) were prepared:
1. L. plantarum (Lactiplantibacillus plantarum) DSM 33370, DSM 33363 and DSM 33364, L. paracasei (Lacticaseibacillus paracasei) DSM 33373 L. brevis (Levilactobacillus brevis) DSM 33377, Bacillus pumilus DSM 33297, DSM 33355, DSM 33301;
2. L. plantarum (Lactiplantibacillus plantarum) DSM 33362 and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300;
3. L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353;
4. L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301;
5. L. brevis (Levilactobacillus brevis) DSM 33377, Pediococcus pentosaceus DSM 33371, L. plantarum (Lactiplantibacillus plantarum) DSM 33369, Bacillus pumilus DSM 33297 and Bacillus megaterium DSM 33300;
6. L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354;
7. L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353.
8. L. plantarum (Lactiplantibacillus plantarum) DSM 33363, DSM 33364, L. paracasei (Lacticaseibacillus paracasei) DSM 33375, L. reuteri (Limosilactobacillus reuteri) DSM 33374, B. megaterium DSM 33300, B. pumilus DSM 33297;
9. L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354;
10. L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356;
11. L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353;
12. L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355;
13. L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297;
14. L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354;
15. L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, B. subtilis DSM 33353;
16. L. plantarum (Lactiplantibacillus plantarum) DSM 33363, DSM 33364, L. paracasei (Lacticaseibacillus paracasei) DSM 33373, L. reuteri (Limosilactobacillus reuteri) DSM 33374, B. megaterium DSM 33300, B. pumilus DSM 33297, DSM 33355.

Five grams of wheat bread (chewed for 30 s and collected in a beaker with 10 mL of NaK-phosphate 0.05 M, pH 6.9) or related dough were suspended in simulated gastric juice containing NaCl (125 mM), KCl (7 mM), NaHCO3 (45 mM), and pepsin (3 g/L) (Sigma-Aldrich CO., St. Louis, Mo., USA). The suspension was added of the pooled selected strains as live (with a final cell density of approximately 9.0 log CFU/mL) and lysed bacteria (corresponding to 9.0 log cells/mL). The calculated initial amount of gluten in the reaction mixture was 7.000 ppm. A control dough, without addition of bacterial mixture, was also subjected to simulated digestion. The suspension was incubated at 37° C., under stirring to simulate peristalsis. After 180 min of gastric digestion, the suspension was added with simulated intestinal fluid, which contained 0.1% (w/v) pancreatin and 0.15% (w/v) Oxgall bile salt (Sigma-Aldrich Co.) at pH 8.0. Besides pancreatin and bile salt, the fluid contained enzymatic preparation E1, E2 (each at 0.2 g/kg), Veron HPP (10 g/100 kg of protein) and Veron PS (g/100 kg of protein) enzymes. Proteases of Aspergillus oryzae (500,000 haemoglobin units on the tyrosine basis/g; enzyme 1 [E1]) and Aspergillus niger (3,000 spectrophotometric acid protease units/g; enzyme 2 [E2]), routinely used for bakery applications, were supplied by BIO-CAT Inc. (Troy, Va.). Veron HPP and Veron PS are bacterial proteases from Bacillus subtilis (AB Enzymes). Enzymatic mixture (E1, E2, Veron PS, Veron HPP) was not added in the control dough.

Intestinal digestion was carried out for 48 h at 37° C. under stirring conditions (ca. 200 rpm). After digestion, samples were put on ice and the concentration of hydrolysed gluten was determined according to a AOAC (Association of Official Agricultural Chemists) International Official Method of Analysis (OMA) (Method No. AACCI 38-55.01) using R5 antibody-based sandwich and competitive ELISA (R5-ELISA) [22]. R5-ELISA analysis was carried out with the RIDASCREEN® Gliadin competitive detection kit according to the instructions of the manufacturer (R-Biopharm AG, Germany). Moreover, ELISA Systems Gluten Residue Detection Kit (Windsor, Australia) was used for quantification of residual gluten. The presence of epitopes in digested samples was monitored after 6, 16, 24, 36 and 48 h of incubation through HPLC analysis. Liquid chromatography coupled with nano electrospray ionization-ion trap tandem mass spectrometry (nano-ESI-MS/MS) was also used to confirm the hydrolysis of gluten and the absence of toxic epitopes.

As estimated by the R5-ELISA (AOAC Official Method of Analysis, Method No. AACCI 38-55.01), after 6 h of digestion the concentration of hydrolysed gluten was in the range of 810±0.02 ppm for the control and 310±0.06 ppm for mixture 3 (Table 2). After 16 and 24 h of digestion, gluten content was 100 ppm for most of the mixtures, with the exception of mixture 16. Importantly, gluten fragment levels were below 20 ppm after 36 h of digestion with mixtures 4 and 16; while gluten fragments were completely absent at the end of incubation (48 h) for mixture 4, 5, 6, 8, and 16.

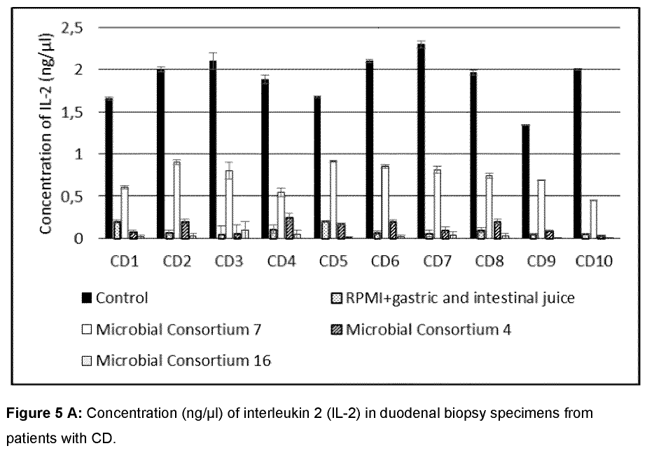

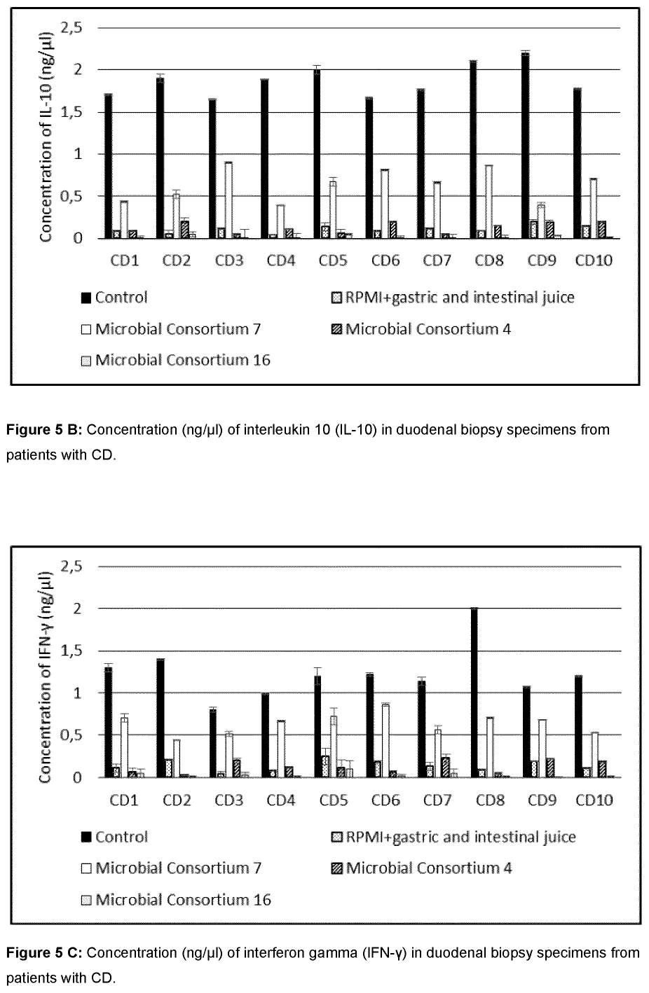

Regarding the residual gluten, most of the mixtures (MC1-9, 16) reduced it below the critical threshold of 20 ppm within 24 h of digestion. Furthermore, mixtures 4-9 and 16 were able to decrease residual gluten to ≤20 ppm within 16 h. Most importantly, the mixture 4 showed complete after 16 h of digestion (FIG. 5). MC8 and MC16 resulted in complete gluten degradation already within the first six hours of digestion. In total, MC4, MC8, and MC16 caused the most efficient removal of intact as well as fragmented gluten (Table 2).

TABLE 2
Concentration (ppm) of residual gluten and peptide fragments of prolamins after 6, 16, 24, 36 and 48 h of simulated gastrointestinal
digestion, as estimated by a specific ELISA tests. Control: dough digested without bacterial cells and commercial enzymes; MC1-MC16:
Microbial consortia constructed by using live and lysed cells of selected Lactobacillus (L.) and Bacillus (B.)
strains and E1, E2, Veron PS, Veron HPP commercial enzymes. Data are the mean of three independent analyses.

| | | Sandwich ELISA assay (Residual gluten) | | | | | Competitive ELISA assay (Gluten fragments) | | | | |
|---|---|---|---|---|---|---|---|---|---|---|---|
| | Strains | 6 h | 16 h | 24 h | 36 h | 48 h | 6 h | 16 h | 24 h | 36 h | 48 h |
| Control | | 1100^{a} ± | 620^{a} ± | 367^{a} ± | 256^{a} ± | 75^{a} ± | 810^{a} ± | 400^{a} ± | 397^{a} ± | 381^{a} ± | 375^{a} ± |
| | | 0.06 | 0.09 | 0.05 | 0.04 | 0.06 | 0.03 | 0.02 | 0.08 | 0.07 | 0.05 |
| MC1 | L. plantarum DSM33370, DSM33363, | 406^{b} ± | 135^{b} ± | 19^{e} ± | 0^{e} | 0^{e} | 310^{f} ± | 250^{d} ± | 200^{e} ± | 170^{e} ± | 65^{g} ± |
| | DSM33364; L. paracasei DSM33373; | 0.04 | 0.06 | 0.01 | | | 0.05 | 0.03 | 0.04 | 0.02 | 0.01 |
| | L. brevis DSM33377; B. pumilus | | | | | | | | | | |
| | DSM33297, DSM33355, DSM33301 | | | | | | | | | | |
| MC2 | L. plantarum DSM33362, DSM33367, | 346^{c} ± | 121^{b} ± | 15^{e} ± | 0^{e} | 0^{e} | 332^{f} ± | 226^{ef} ± | 167^{f} ± | 158^{e} ± | 150^{c} ± |
| | DSM33368; L. paracasei DSM33375; | 0.07 | 0.03 | 0.01 | | | 0.05 | 0.04 | 0.03 | 0.02 | 0.02 |
| | B. subtilis DSM33298; B. | | | | | | | | | | |
| | licheniformis DSM33354; B. | | | | | | | | | | |
| | megaterium DSM33300 | | | | | | | | | | |
| MC3 | L. plantarum DSM33366, DSM33369; | 382^{c} ± | 99^{c} ± | 12^{f} ± | 0^{e} | 0^{e} | 315^{f} ± | 272^{d} ± | 256^{d} ± | 244^{c} ± | 228^{b} ± |
| | L. reuteri DSM33374; L. paracasei | 0.03 | 0.02 | 0.01 | | | 0.06 | 0.07 | 0.04 | 0.05 | 0.02 |
| | DSM33376; P. pentosaceus | | | | | | | | | | |
| | DSM33371; B. megaterium | | | | | | | | | | |
| | DSM33356; B. subtilis DSM33353 | | | | | | | | | | |
| MC4 | L. plantarum DSM33363, DSM33364; | 190^{cd} ± | 0^{g} | 0^{g} | 0^{e} | 0^{e} | 399^{e} ± | 233^{e} ± | 112^{g} ± | 0^{j} | 0^{g} |
| | L. paracasei DSM33373; B. subtilis | 0.05 | | | | | 0.08 | 0.07 | 0.05 | | |
| | DSM33298; B. pumilus DSM33301 | | | | | | | | | | |
| MC5 | L. brevis DSM33377; P. pentosaceus | 380^{b} ± | 18^{g} ± | 5^{g} ± | 0^{e} | 0^{e} | 398^{e} ± | 221^{e} ± | 154^{fg} ± | 46ⁱ ± | 0^{g} |
| | DSM33371; L. plantarum DSM33369; | 0.06 | 0.01 | 0.01 | | | 0.04 | 0.05 | 0.03 | 0.02 | |
| | B. pumilus DSM33297; B. megaterium | | | | | | | | | | |
| | DSM33300 | | | | | | | | | | |
| MC6 | L. paracasei DSM33375; L. plantarum | 350^{c} ± | 15^{e} ± | 2^{g} ± | 0^{e} | 0^{e} | 404^{e} ± | 245^{de} ± | 100^{g} ± | 79^{h} ± | 0^{g} |
| | DSM33367, DSM33368; B. pumilus | 0.06 | 0.02 | 0.01 | | | 0.06 | 0.05 | 0.08 | 0.04 | |
| | DSM33355; B. licheniformis | | | | | | | | | | |
| | DSM33354 | | | | | | | | | | |
| MC7 | L. plantarum DSM33370, DSM33362, | 360^{c} ± | 20^{e} ± | 10^{f} ± | 0^{e} | 0^{e} | 401^{e} ± | 261^{d} ± | 150^{f} ± | 99^{g} ± | 78^{g} ± |
| | DSM33366; L. reuteri DSM33374; B. | 0.09 | 0.06 | 0.01 | | | 0.07 | 0.05 | 0.03 | 0.04 | 0.05 |
| | megaterium DSM33356; B. subtilis | | | | | | | | | | |
| | DSM33353 | | | | | | | | | | |
| MC8 | L. plantarum DSM33363, DSM33364; | 18^{g} ± | 3^{g} ± | 0^{g} | 0^{e} | 0^{e} | 323^{f} ± | 228^{e} ± | 218^{e} ± | 157^{e} ± | 0^{g} |
| | L. paracasei DSM33375; L. reuteri | 0.03 | 0.01 | | | | 0.08 | 0.06 | 0.05 | 0.06 | |
| | DSM33374; B. megaterium | | | | | | | | | | |
| | DSM33300; B. pumilus DSM33297 | | | | | | | | | | |
| MC9 | L. paracasei DSM33375; L. plantarum | 60^{f} ± | 12^{f} ± | 0^{g} | 0^{e} | 0^{e} | 319^{f} ± | 211^{f} ± | 196^{ef} ± | 195^{de} ± | 152^{c} ± |
| | DSM33367; L. reuteri DSM33374; B. | 0.04 | 0.01 | | | | 0.06 | 0.05 | 0.03 | 0.07 | 0.02 |
| | megaterium DSM33300; B. pumilus | | | | | | | | | | |
| | DSM33297; B. licheniformis | | | | | | | | | | |
| | DSM33354 | | | | | | | | | | |
| MC10 | L. plantarum DSM33363, DSM33364, | 112^{e} ± | 77^{d} ± | 70^{d} ± | 0^{e} | 0^{e} | 465^{d} ± | 370^{b} ± | 243^{de} ± | 145^{ef} ± | 97^{f} ± |
| | DSM33370; L. brevis DSM33377; B. | 0.06 | 0.04 | 0.02 | | | 0.09 | 0.06 | 0.05 | 0.04 | 0.03 |
| | pumilus DSM33297; B. megaterium | | | | | | | | | | |
| | DSM33356 | | | | | | | | | | |
| MC11 | L. plantarum DSM33368, DSM33362, | 221^{d} ± | 89^{c} ± | 69^{d} ± | 50^{d} ± | 43^{d} ± | 512^{c} ± | 367^{b} ± | 340^{b} ± | 300^{b} ± | 123^{de} ± |
| | DSM33367; L. paracasei DSM33375; | 0.05 | 0.07 | 0.06 | 0.04 | 0.03 | 0.06 | 0.08 | 0.09 | 0.06 | 0.05 |
| | B. megaterium DSM33300; B. subtilis | | | | | | | | | | |
| | DSM33353 | | | | | | | | | | |
| MC12 | L. plantarum DSM33366, DSM33369; | 145^{e} ± | 110^{c} ± | 89^{c} ± | 75^{c} ± | 63^{b} ± | 601^{b} ± | 312^{c} ± | 289^{c} ± | 288^{b} ± | 143^{ed} ± |
| | L. reuteri DSM33374; L. paracasei | 0.06 | 0.05 | 0.03 | 0.02 | 0.03 | 0.09 | 0.06 | 0.07 | 0.05 | 0.03 |
| | DSM33376; P. pentosaceus | | | | | | | | | | |
| | DSM33371; B. pumilus DSM33297, | | | | | | | | | | |
| | DSM33355 | | | | | | | | | | |
| MC13 | L. brevis DSM33377; P. pentosaceus | 163^{de} ± | 122^{b} ± | 82^{c} ± | 45^{d} ± | 0^{e} | 523^{c} ± | 322^{c} ± | 321^{b} ± | 215^{d} ± | 134^{d} ± |
| | DSM33371; L. sanfranciscensis | 0.06 | 0.04 | 0.02 | 0.03 | | 0.07 | 0.07 | 0.06 | 0.07 | 0.05 |
| | DSM33379; B. megaterium | | | | | | | | | | |
| | DSM33300; B. pumilius DSM33297 | | | | | | | | | | |
| MC14 | L. plantarum DSM33368; L. paracasei | 234^{d} ± | 135^{b} ± | 120^{b} ± | 108^{b} ± | 56^{c} ± | 587^{b} ± | 333^{c} ± | 256^{d} ± | 211^{d} ± | 167^{c} ± |
| | DSM33375; L. sanfranciscensis | 0.08 | 0.07 | 0.07 | 0.05 | 0.03 | 0.09 | 0.09 | 0.08 | 0.08 | 0.07 |
| | DSM33378; B. megaterium | | | | | | | | | | |
| | DSM33300; B. pumilus DSM33297; | | | | | | | | | | |
| | B. licheniformis DSM33354 | | | | | | | | | | |
| MC15 | L. plantarum DSM33362, DSM33366, | 199^{d} ± | 100^{c} ± | 81^{c} ± | 59^{d} ± | 40^{d} ± | 498^{c} ± | 318^{c} ± | 280^{c} ± | 256^{c} ± | 118^{e} ± |
| | DSM33370; L. reuteri DSM33374; L. | 0.05 | 0.04 | 0.05 | 0.04 | 0.03 | 0.08 | 0.04 | 0.03 | 0.08 | 0.05 |
| | sanfranciscensis DSM33378, | | | | | | | | | | |
| | DSM33379; B. licheniformis | | | | | | | | | | |
| | DSM33354; B. subtilis DSM33353 | | | | | | | | | | |
| MC16 | L. plantarum DSM33363, DSM33364; | 19^{g} ± | 11^{f} ± | 0^{g} | 0^{e} | 0^{e} | 280^{g} ± | 200^{f} ± | 50^{h} ± | 10^{j} ± | 0^{g} |
| | L. paracasei DSM33373; L. reuteri | 0.03 | 0.01 | | | | 0.06 | 0.05 | 0.03 | 0.01 | |
| | DSM33374; B. megaterium | | | | | | | | | | |
| | DSM33300; B. pumilus DSM33297, | | | | | | | | | | |
| | DSM33355 | | | | | | | | | | |

^{a-j}Values with different superscript letters, in the same row, differ significantly (P < 0.05).

Based on the calculated initial amount of gluten in the reaction mixture of 7.000 ppm, regarding the residual gluten, all the mixtures were able to reduce it by at least 94% after 6 h (in comparison to a reduction of around 84% for the control), by at least 98% after 16 h and up to at least 99.1% after 48 h. Regarding gluten fragments, those were reduced by all mixtures by at least 91% after 6 h (in comparison to a reduction of around 88% for the control), by at least 95% after 16 h and up to at least 97% after 48 h.

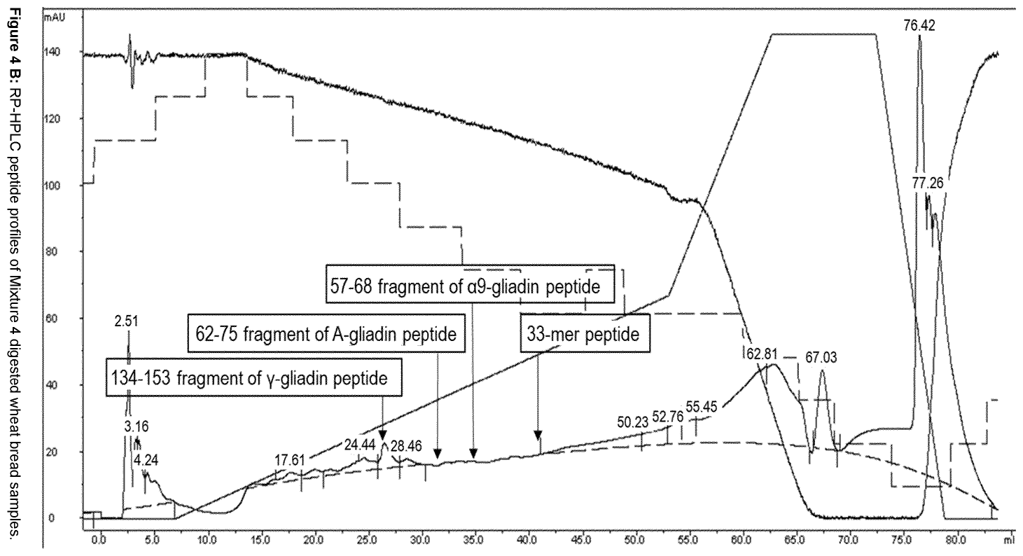

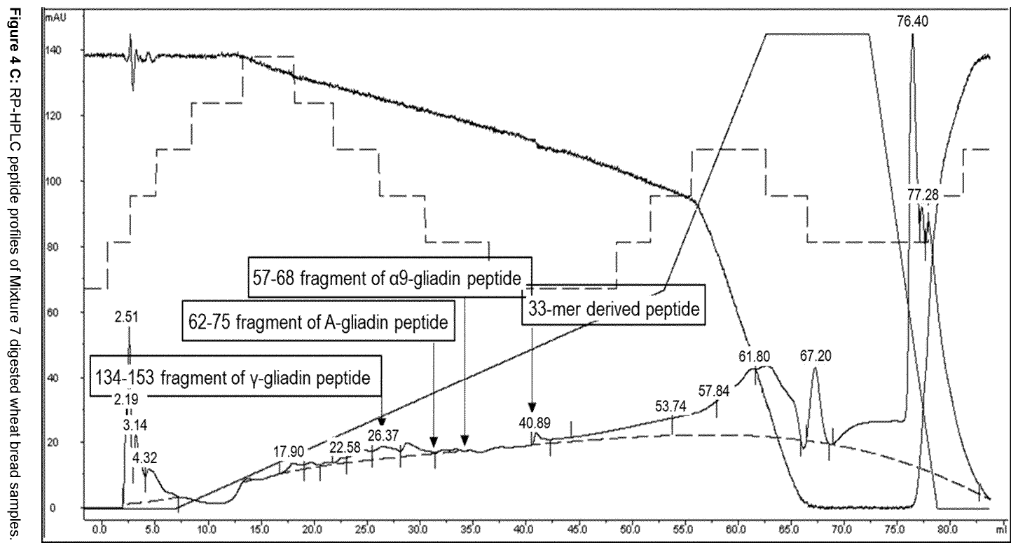

Regarding the residual gluten, the most efficient strains MC4, MC8, and MC16 were able to reduce it by at least 97% after 6 h, at least 99.8% after 16 h and up to 100% after 24 h. Regarding the gluten fragments, those were reduced by the most efficient strains MC4, MC8, and MC16 by at least 94% after 6 h, by at least 97% after 16 h, by at least 98% after 36 h and to 100% after 48 h. FIG. 4 shows RP-HPLC peptide profiles of control (panel A), Mixture 4 (panel B) and Mixture 7 (panel C) digested wheat bread samples. M4 and M7 were combined with of E1, E2, Veron PS, Veron HPP commercial enzymes. Mixture 4 led to full (93%) hydrolysis of all immunogenic peptides, whereas only partial (56%) hydrolysis was achieved by Mixture 7. In conclusion, we have found fully functional mixtures comprising only 4-7 selected strains, as compared to the more extensive mixtures disclosed in Example 3.

For exemplary microbial consortia we performed experiments with and without added commercial enzymes. The consortia alone led to strong reductions of residual as well as hydrolysed gluten, and this was further enhanced by added enzymes.

### Example 5. Assessment of Immunogenicity of Gluten Digests by Using Duodenal Explants from Celiac Disease Patients

Immunogenicity of the digests was ex vivo estimated by testing the cytokine expression in duodenal biopsy specimens from patients with celiac disease (CD). All CD patients expressed the HLA-DQ2 phenotype. CD was diagnosed according to European Society for Paediatric Gastroenterology, Hepatology, and Nutrition criteria [23]. Immediately after excision, all biopsy specimens were placed in ice-chilled culture medium (RPMI 1640; Gibco-Invitrogen, UK) and transported to the laboratory within 30 min. Duodenal biopsy specimens were cultured for 4 h using the organ tissue culture method originally described by Browning and Trier [24]. Briefly, the biopsy specimens were oriented villous side up on a stainless-steel mesh and positioned over the central well of an organ tissue culture dish (Falcon, USA). The well contained RPMI supplemented with 15% foetal calf serum (Gibco-Invitrogen) and 1% penicillin-streptomycin (Gibco-lnvitrogen, UK). Dishes were placed into an anaerobic jar and incubated at 37° C.

Digested samples of control dough (positive control) (wheat bread digested without the addition of bacterial cells and microbial enzymes), Mixture 4 (wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes) and Mixture 7 (wheat bread digested with the addition of live and lysed cells of L. plantarum (Lactiplantibacillus plantarum) DSM 33362, and DSM 33366, Lactobacillus reuteri (Limosilactobacillus reuteri) DSM 33374, L. plantarum (Lactiplantibacillus plantarum) DSM 33370, Bacillus megaterium DSM 33356, and Bacillus subtilis DSM 33353 and E1, E2, Veron PS, Veron HPP commercial enzymes) were subjected to gliadin and glutenin polypeptide extraction and used for assessing their ability to induce cytokine expression in duodenal biopsy specimens from CD patients. Four biopsy specimens from each CD patient were cultured with culture medium under five conditions: (i) with doughs containing the Mixture 4 (wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes) digested for 48 h; (ii) with dough containing Mixture 7 (wheat bread digested with the addition of live and lysed cells of L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353 and E1, E2, Veron PS, Veron HPP commercial enzymes) digested for 48 h; (iii) with dough containing Mixture 16 (wheat bread digested with the addition of live and lysed cells of L. plantarum (Lactiplantibacillus plantarum) DSM 33363 and DSM 33364, Lactobacillus reuteri (Limosilactobacillus reuteri) DSM 33374, Bacillus megaterium DSM 33330, and Bacillus pumilus DSM 33297 and DSM 33355 and E1, E2, Veron PS, Veron HPP commercial enzymes) digested for 48 h; (iv) with control dough digested for 48 h (Control); and (v) with culture medium (RPMI 1640+gastric and intestinal juice, negative control). Biopsy specimens from each patient were rinsed and stored in RNAlater (Qiagen GmbH, Germany) at −80° C. to preserve the RNA. Total RNA was extracted from the tissues using the RNeasy minikit (Qiagen GmbH) according to the manufacturer's instructions. The concentration of mRNA was estimated by determination of the UV absorbance at 260 nm. Aliquots of total RNA (500 ng) were reverse transcribed using random hexamers, TaqMan reverse transcription reagents (Applied Biosystems, Monza, Italy), and 3.125 U/μl of MultiScribe reverse transcriptase to a final volume of 50 μl. The cDNA samples were stored at −20° C. RT-PCR was performed in 96-well plates using an ABI Prism 7500HT fast sequence detection system (Applied Biosystems). Data collection and analyses were performed using the machine software. PCR primers and fluorogenic probes for the target genes (IFN-γ, IL-2, and IL-10) and the endogenous control (gene coding for glyceraldehyde-3-phosphate dehydrogenase [GAPDH]) were purchased as a TaqMan gene expression assay and a pre-developed TaqMan assay (Applied Biosystems), respectively. The assays were supplied as a 20× mix of PCR primers and TaqMan Minor Groove Binder 6-carboxyfluorescein dye-labelled probes with a non-fluorescent quencher at the 3′ end of the probe. Two-step reverse transcription-PCR was performed using first-strand cDNA with a final concentration of 1× TaqMan gene expression assay mix and 1× TaqMan universal PCR master mix. The final reaction volume was 25 μl. Each sample was analysed in triplicate, and all experiments were repeated twice. A non-template control (RNase-free water) was included with every plate. The following thermal cycler conditions were used: 2 min at 50° C. (uracil DNA glycosylase activation), 10 min at 95° C., and 40 cycles of 15 s at 95° C. and 1 min at 60° C. Initially, a standard curve and a validation experiment were performed for each primer/probe set. Six serial dilutions (20 to 0.1 ng/μl) of IFN-γ, IL-2, or IL-10 cDNA were used as a template for each primer/probe set. A standard curve was generated by plotting the threshold cycle (CT) values against the log of the amount of input cDNA. The CT value is the PCR cycle at which an increase in reporter fluorescence above the baseline level is first detected. The average value for the target gene was normalized using an endogenous reference gene (the GAPDH gene). A healthy duodenal biopsy specimen was used to calibrate all the experiments. The levels of IFN-γ, IL-2, and IL-10 proteins secreted into the supernatant were quantified by ELISA in 96-well round-bottom plates (Tema Ricerca, Milan, Italy) according to the manufacturer's recommendations.

As expected, the duodenal biopsy specimens incubated with positive control produced significantly (P<0.05) higher expression of interleukin 2 (IL-2), interleukin 10 (IL-10) (B), and interferon gamma (IFN-γ) mRNA than the negative control (RPMI 1640+gastric and intestinal juice) (FIG. 5). Compared to negative control, the samples digested with the Mixtures 4 and 16 showed the same (P>0.05) level of IL-2, IL-10 and IFN-γ. The Mixture 7 was characterized by lower synthesis of IL-2 than the positive control, but, compared to the negative control as well as mixtures 4 and 16, by higher synthesis of IL-2. Similar trends were also found for IL-10 and IFN-γ. These results correlate nicely with the full and partial clearance of immunogenic peptides by mixture 4 and 7, respectively, as shown in FIG. 4A-C.

FIG. 5A shows concentration (ng/pl) of interleukin 2 (IL-2) in duodenal biopsy specimens from patients with CD. Control: wheat bread digested without the addition of bacterial cells and microbial enzymes; RPMI+gastric and intestinal juice: negative control; Microbial Consortium 4: wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes); Microbial Consortium 7: wheat bread digested with the addition of live and lysed cells of L. plantarum (Lactiplantibacillus plantarum) DSM 33362, DSM 33366 and DSM 33370, L. reuteri (Limosilactobacillus reuteri) DSM 33374, Bacillus megaterium DSM 33356, and Bacillus subtilis DSM 33353 and E1, E2, Veron PS, Veron HPP commercial enzymes; and Microbial Consortium 16: wheat bread digested with the addition of live and lysed cells of L. plantarum (Lactiplantibacillus plantarum) DSM 33363, DSM 33364, L. paracasei (Lacticaseibacillus paracasei) DSM 33373, L. reuteri (Limosilactobacillus reuteri) DSM 33374, Bacillus megaterium DSM 33330, Bacillus pumilus DSM 33297, DSM 33355. CD1 to CD10, duodenal biopsy specimens from celiac patients.

FIG. 5B shows concentration (ng/μl) of interleukin 10 (IL-10) in duodenal biopsy specimens from patients with CD. Samples and microbial consortia are equivalent to FIG. 5A.

FIG. 5C shows concentration (ng/μl) of interferon gamma (IFN-γ) in duodenal biopsy specimens from patients with CD. Samples and microbial consortia are equivalent to FIG. 5A.

The findings of this invention provide evidence that the selected combinations of probiotic bacterial strains have the potential to improve the digestion of gluten in gluten-sensitive patients and to hydrolyse immunogenic peptides during gastrointestinal digestion, which decreases gluten toxicity for gluten-sensitive patients in general, and for CD patients particularly.

### REFERENCES

1. Sapone A, Bai J C, Ciacci C, Dolinsek J, Green P H, Hadjivassiliou M, Kaukinen K, Rostami K, Sanders D S, Schumann M et al: Spectrum of gluten-related disorders: consensus on new nomenclature and classification. BMC Med 2012, 10:13.
2. Niland B, Cash B D: Health Benefits and Adverse Effects of a Gluten-Free Diet in Non-Celiac Disease Patients. Gastroenterol Hepatol (N Y) 2018, 14(2):82-91.
3. Lis D M, Stellingwerff T, Shing C M, Ahuja K D, Fell J W: Exploring the popularity, experiences, and beliefs surrounding gluten-free diets in nonceliac athletes. Int J Sport Nutr Exerc Metab 2015, 25(1):37-45.
4. Wild D, Robins G G, Burley V J, Howdle P D: Evidence of high sugar intake, and low fibre and mineral intake, in the gluten-free diet. Aliment Pharmacol Ther 2010, 32(4):573-581
5. Thompson T, Dennis M, Higgins L A, Lee A R, Sharrett M K: Gluten-free diet survey: are

Americans with coeliac disease consuming recommended amounts of fibre, iron, calcium and grain foods? J Hum Nutr Diet 2005, 18(3):163-169.
6. Larretxi I, Simon E, Benjumea L, Miranda J, Bustamante M A, Lasa A, Eizaguirre F J, Churruca I: Gluten-free-rendered products contribute to imbalanced diets in children and adolescents with celiac disease. Eur J Nutr 2018.
7. De Palma G, Nadal I, Collado M C, Sanz Y: Effects of a gluten-free diet on gut microbiota and immune function in healthy adult human subjects. Br J Nutr 2009, 102(8):1154-1160.
8. Galipeau H J, McCarville J L, Huebener S, Litwin O, Meisel M, Jabri B, Sanz Y, Murray J A, Jordana M, Alaedini A et al: Intestinal microbiota modulates gluten-induced immunopathology in humanized mice. Am J Pathol 2015, 185(11):2969-2982.
9. Caminero A, Galipeau H J, McCarville J L, Johnston C W, Bernier S P, Russell A K, Jury J, Herran A R, Casqueiro J, Tye-Din J A et al: Duodenal Bacteria From Patients With Celiac Disease and Healthy Subjects Distinctly Affect Gluten Breakdown and Immunogenicity. Gastroenterology 2016, 151(4):670-683.
10. Francavilla R, Ercolini D, Piccolo M, Vannini L, Siragusa S, De Filippis F, De Pasquale I, Di Cagno R, Di Toma M, Gozzi G et al: Salivary microbiota and metabolome associated with celiac disease. Appl Environ Microbiol 2014, 80(11):3416-3425.
11. De Angelis M, Cassone A, Rizzello C G, Gagliardi F, Minervini F, Calasso M, Di Cagno R, Francavilla R, Gobbetti M: Mechanism of degradation of immunogenic gluten epitopes from Triticum turgidum L. var. durum by sourdough lactobacilli and fungal proteases. Appl Environ Microbiol 2010, 76(2):508-518.
12. Francavilla R, Cristofori F, Verzillo L, Gentile A, Castellaneta S, Polloni C, Giorgio V, Verduci E, D'Angelo E, Dellatte S et al: Randomized Double-Blind Placebo-Controlled Crossover Trial for the Diagnosis of Non-Celiac Gluten Sensitivity in Children. Am J Gastroenterol 2018, 113(3):421-430.
13. Francavilla R, De Angelis M, Rizzello C G, Cavallo N, Dal Bello F, Gobbetti M: Selected Probiotic Lactobacilli Have the Capacity To Hydrolyze Gluten Peptides during Simulated Gastrointestinal Digestion. Appl Environ Microbiol 2017, 83(14).
14. Herran A R, Perez-Andres J, Caminero A, Nistal E, Vivas S, Ruiz de Morales J M, Casqueiro J: Gluten-degrading bacteria are present in the human small intestine of healthy volunteers and celiac patients. Res Microbiol 2017, 168(7):673-684.
15. Caminero A, Herran A R, Nistal E, Perez-Andres J, Vaquero L, Vivas S, Ruiz de Morales J M, Albillos S M, Casqueiro J: Diversity of the cultivable human gut microbiome involved in gluten metabolism: isolation of microorganisms with potential interest for coeliac disease. FEMS Microbiol Ecol 2014, 88(2):309-319.
16. Pyle G G, Paaso B, Anderson B E, Allen D D, Marti T, Li Q, Siegel M, Khosla C, Gray G M: Effect of pretreatment of food gluten with prolyl endopeptidase on gluten-induced malabsorption in celiac sprue. Clin Gastroenterol Hepatol 2005, 3(7):687-694.
17. Krishnareddy S, Stier K, Recanati M, Lebwohl B, Green P H: Commercially available glutenases: a potential hazard in coeliac disease. Therap Adv Gastroenterol 2017, 10(6):473-481.
18. Shan L, Marti T, Sollid L M, Gray G M, Khosla C: Comparative biochemical analysis of three bacterial prolyl endopeptidases: implications for coeliac sprue. Biochem J 2004, 383(Pt 2):311-318.
19. Fernandez M F, Boris S, Barbes C: Probiotic properties of human lactobacilli strains to be used in the gastrointestinal tract. J Appl Microbiol 2003, 94(3):449-455.
20. Zarate G, Chaia A P, Gonzalez S, Oliver G: Viability and beta-galactosidase activity of dairy propionibacteria subjected to digestion by artificial gastric and intestinal fluids. J Food Prot 2000, 63(9):1214-1221.
21. De Angelis M, Siragusa S, Berloco M, Caputo L, Settanni L, Alfonsi G, Amerio M, Grandi A, Ragni A, Gobbetti M: Selection of potential probiotic lactobacilli from pig feces to be used as additives in pelleted feeding. Res Microbiol 2006, 157(8):792-801.
22. Valdes I, Garcia E, Llorente M, Mendez E: Innovative approach to low-level gluten determination in foods using a novel sandwich enzyme-linked immunosorbent assay protocol. Eur J Gastroenterol Hepatol 2003, 15(5):465-474.
23. of ES: Revised criteria for diagnosis of coeliac disease. Report of Working Group of European Society of Paediatric Gastroenterology and Nutrition. Arch Dis Child 1990, 65(8):909-911.
24. Browning T H, Trier J S: Organ culture of mucosal biopsies of human small intestine. J Clin Invest 1969, 48(8):1423-1432.

1. A preparation comprising a consortia of at least one bacterial strain selected from the genus Bacillus and at least one bacterial strain selected from the genus Lactobacillus, for use in the degradation of gluten to a gluten content of 20 ppm or less,
wherein the consortia of strains can degrade the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), and the 33-mer peptide LQLQPFPQPQLPYPQPQLPYPQPQLPYPQPQPF (Seq-ID No 4).

2. The preparation according to claim 1, wherein the consortia of strains can degrade the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), and the 33-mer peptide LQLQPFPQPQLPYPQPQLPYPQPQLPYPQPQPF (Seq-ID No 4) by at least 80%.

3. The preparation according to claim 1, wherein the consortia of strains can degrade the gluten to a digest that does not cause an immunogenic or toxic response in a small intestine or a small intestinal explant of a subject or an animal affected by a gluten-related disorder, and/or
wherein all strains of the consortia survive (less than 2 log CFU loss) in simulated gastric (pH 1.0-4.0) and intestinal (pH 5.4-6.8, 0.05-0.6% bile acids, or a concentration of bile acids present in humans under in vivo conditions) conditions, and/or
wherein members of the consortia have complementary PepP, PepO, PepX, PepI, and PepN activities with at least one peptidase activity equal to or more than 3 U/g (PepP), 5 U/g (PepO), 20 U/g (PepX), 17 U/g (PepI), and 20 U/g (PepN).

4. The preparation according to claim 1, wherein the Bacillus strains are selected from the group consisting of Bacillus pumilus, Bacillus subtilis, Bacillus licheniformis, and Bacillus megaterium.

5. The preparation according to claim 1, wherein the Lactobacillus strains are selected from the group consisting of Lactobacillus plantarum (Lactiplantibacillus plantarum), Lactobacillus casei (Lacticaseibacillus casei), Lactobacillus paracasei (Lacticaseibacillus paracasei), Lactobacillus brevis (Levilactobacillus brevis), Lactobacillus sanfranciscensis (Fructilactobacillus sanfranciscensis), and Lactobacillus reuteri (Limosilactobacillus reuteri).

6. The preparation according to claim 1, wherein the probiotic strains are present in a dormant form or as vegetative cells.

7. The preparation according to claim 1, wherein cytoplasmic extracts or cell-free supernatants or heat-killed biomass of the strains are used.

8. The preparation according to claim 1, wherein the preparation further comprises at least one selected from the group consisting of Pediococcus sp., and Weissella sp.

9. The preparation according to claim 1, wherein the preparation further comprises one or more microbial proteases purified from at least one selected from the group consisting of Aspergillus niger, Aspergillus oryzae, Bacillus sp., Lactobacillus sp., Pediococcus sp., Weissella sp., Rothia niucilaginosa, Rothia aeria, subtilisins, nattokinase, arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum, xylooligosaccharides, and alginate.

10. The preparation according to claim 1 for treating or preventing gluten-related disorders, wherein the disorder is at least one selected from the group consisting of celiac disease, non-celiac gluten sensitivity, wheat allergy, and gluten-sensitive irritable bowel syndrome in a subject or an animal in need thereof.

11. The preparation according to claim 1 for producing gluten-free foods from at least one gluten-containing cereal selected from the group consisting of wheat, barley, rye, and oat.

12. The preparation according to claim 1, further comprising a substance, which acts as a permeabilizer of a microbial cell membrane of at least one selected from the group consisting of Bacillus sp., Lactobacillus sp., Pediococcus sp., and Weissella sp.

13. The preparation according to claim 1, wherein the strains are selected from Bacillus sp., Lactobacillus sp., Pediococcus sp., and Weissella sp., and are immobilized individually or as consortia.

14. A preparation, comprising at least one strain of each of the following groups 1-5:
Group 1: Lactobacillus paracasei (Lacticaseibacillus paracasei) DSM 33373, Lactobacillus paracasei (Lacticaseibacillus paracasei) DSM 33375,
Group 2: Bacillus subtilis DSM 33298, Bacillus pumilus DSM 33297, Bacillus licheniformis DSM 33354, Bacillus megaterium DSM 33356, Pediococcus pentosaceus DSM 33371, Lactobacillus plantarum (Lactiplantibacillus plantarum) DSM 33370, Lactobacillus brevis (Levilactobacillus brevis) DSM 33377, Lactobacillus paracasei (Lacticaseibacillus paracasei) DSM 33376, Lactobacillus paracasei (Lacticaseibacillus paracasei) DSM 33375, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33363, Lactobacillus paracasei (Lacticaseibacillus paracasei) DSM 33373, Lactobacillus plantarum (Lactiplantibacillus plantarum) DSM 33362,
Group 3: Lactobacillus sanfranciscensis (Fructilactobacillus sanfranciscensis) DSM 33378, Lactobacillus sanfranciscensis (Fructilactobacillus sanfranciscensis) DSM 33379, Pediococcus pentosaceus DSM 33371, Lactobacillus plantarum (Lactiplantibacillus plantarum) DSM 33370, Lactobacillus plantarum (Lactiplantibacillus plantarum) DSM 33369, Lactobacillus reuteri (Limosilactobacillus reuteri) DSM 33374, Lactobacillus plantarum (Lactiplantibacillus plantarum) DSM 33363, Lactobacillus paracasei (Lacticaseibacillus paracasei) DSM 33373,
Group 4: Bacillus subtilis DSM 33353, Bacillus pumilus DSM 33355, Bacillus pumilus DSM 33301,
Group 5: Bacillus megaterium DSM 33300, Lactobacillus sanfranciscensis (Fructilactobacillus sanfranciscensis) DSM 33378, Pediococcus pentosaceus DSM 33371, Lactobacillus brevis (Levilactobacillus brevis) DSM 33377, Lactobacillus plantarum (Lactiplantibacillus plantarum) DSM 33368, Lactobacillus reuteri (Limosilactobacillus reuteri) DSM 33374, Lactobacillus plantarum (Lactiplantibacillus plantarum) DSM 33367, Lactobacillus plantarum (Lactiplantibacillus plantarum) DSM 33366, Lactobacillus plantarum (Lactiplantibacillus plantarum) DSM 33364, and Lactobacillus paracasei (Lacticaseibacillus paracasei) DSM 33373.

15. The preparation according to claim 14, comprising at least three different strains.

16. The preparation according to claim 14, further comprising the strain selected at least one from the group consisting of following strains:
L. plantarum (Lactiplantibacillus plantarum) DSM 33370, DSM 33363, DSM 33364; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300 and Bacillus subtilis DSM 33353, or
L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298 and DSM 33353, or
L. plantarum (Lactiplantibacillus plantarum) DSM 33366, and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356, and Bacillus subtilis DSM 33298, or
L. plantarum (Lactiplantibacillus plantarum) DSM 33363, and DSM 33364, L. paracasei (Lacticaseibacillus paracasei) DSM 33373, Bacillus subtilis DSM 33298, and Bacillus pumilus DSM 33301, or
L. plantarum (Lactiplantibacillus plantarum) DSM 33363, and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
L. plantarum (Lactiplantibacillus plantarum) DSM 33363, and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355, or
L. plantarum (Lactiplantibacillus plantarum) DSM 33370, DSM 33363, and DSM 33364, L. paracasei (Lacticaseibacillus paracasei) DSM 33373, L. brevis (Levilactobacillus brevis) DSM 33377, Bacillus pumilus DSM 33297, DSM 33355, DSM 33301, or
L. plantarum (Lactiplantibacillus plantarum) DSM 33362, and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300, or
L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353, or
L. brevis (Levilactobacillus brevis) DSM 33377, Pediococcus pentosaceus DSM 33371, L. plantarum (Lactiplantibacillus plantarum) DSM 33369, Bacillus pumilus DSM 33297, and Bacillus megaterium DSM 33300, or
L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354, or
L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353, or
L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354, or
L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356, or
L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353, or
L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355, or
L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297, or
L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354, or
L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, and B. subtilis DSM 33353.

17. A product comprising at least one selected from the group consisting of a food supplement, a pet food supplement, a food product, a pet food product, and a pharmaceutical product comprising the preparation of claim 14.


SEQUENCE LISTING (4 sequences)

SEQ ID NO: 1 - protein, 12 aa, Unknown
  Alpha9-gliadin
QLQPFPQPQLPY

SEQ ID NO: 2 - protein, 14 aa, Unknown
  A-gliadin
PQPQLPYPQPQSFP

SEQ ID NO: 3 - protein, 20 aa, Unknown
  Y-gliadin
QQLPQPQQPQQSFPQQQRPF

SEQ ID NO: 4 - protein, 33 aa, Unknown
  alpha2-gliadin
LQLQPFPQPQLPYPQPQLPYPQPQLPYPQPQPF